(19) United States
(12) Patent Application Publication
(10) Pub. No.: US 2010/0304358 A1
(43) Pub. Date: Dec. 2, 2010

(54) METHODS OF IDENTIFYING BIOLOGICAL TARGETS AND INSTRUMENTATION TO IDENTIFY BIOLOGICAL TARGETS

(76) Inventors: Shuming Nie, Atlanta, GA (US); Amit Agrawal, Medford, MA (US); Zhang Chun Yang, Flushing, NY (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/063,882

(22) PCT Filed: Aug. 15, 2006
(86) PCT No.: PCT/US06/31763

§ 371 Date: Jun. 10, 2010

Related U.S. Application Data
(60) Provisional application No. 60/708,240, filed on Aug. 15, 2005.

(51) Int. Cl. C12Q 1/70; C12Q 1/02; G01J 1/58; G01N 33/50; G01N 33/53

(52) U.S. Cl. 435/5; 435/29; 250/458.1; 436/86; 436/94; 436/71; 250/200

(57) ABSTRACT

Methods of measuring and/or detecting biological targets, methods of distinguishing among the same type of biological target, single-molecule detection systems, fluorescent/biological target complexes, methods of using fluorescent/biological target complexes, and the like are disclosed.

### CROSS-REFERENCE TO RELATED APPLICATION

This application claims priority to copending U.S. provisional patent application entitled “Methods of Counting Single Native Biomolecules and Intact Viruses in Solution” filed on Aug. 15, 2005 and accorded Ser. No. 60/563,865, which is entirely incorporated herein by reference.

### STATEMENT REGARDING FEDERALLY SPONSORED RESEARCH OR DEVELOPMENT

This invention was made with government support under Grant Nos.: P20 GM072069, R01 CA108468-01, and R01 GM60562 awarded by the National Institute of Health. The government has certain rights in the invention.

### BACKGROUND

The detection and identification of single molecules represents the ultimate limit in chemical analysis, medical diagnostics, and in vivo molecular imaging. By removing population averaging, single-molecule measurements also provide new insights into the fundamental mechanisms of ligand-receptor binding, signal transduction, and intracellular transport. Recent advances have allowed direct imaging and dynamic studies of single biomacromolecules (e.g., DNA, RNA, and proteins) as well as multicomponent molecular assemblies (e.g., molecular machines). For applications in chemical analysis and medical diagnostics, however, single-molecule methodologies have achieved only limited success. A major problem is that current organic dyes and fluorescent proteins are not bright enough or stable enough for routine single-molecule analysis. Another problem is that target molecules often need to be chemically derivatized with a fluorophore, a difficult task for low-abundance genes and proteins. A further challenge is the need to discriminate bound targets from excess unbound probes inside single living cells or in complex mixtures.

### SUMMARY

Briefly described, embodiments of this disclosure include methods of measuring and/or detecting biological targets, methods of distinguishing among the same type of biological target, single-molecule detection systems, fluorescent/biological target complexes, methods of using fluorescent/biological target complexes, and the like.

One exemplary method of detecting a biological target, among others, includes providing a first quantum dot and a second quantum dot, wherein the first quantum dot and the second quantum dot emit energy at different wavelengths, wherein the emission from the first quantum dot is at a first wavelength and the emission from the second quantum dot is at a second wavelength, wherein the first wavelength and the second wavelength are detectably distinguishable, wherein the first quantum dot includes a first biomolecule having an affinity for a first binding site of a biological target, wherein the second quantum dot includes a second biomolecule having an affinity for a second binding site of the biological target, and wherein the biological target includes the first binding site and the second binding site; mixing the first quantum dot and the second quantum dot with a sample, wherein if the sample includes the biological target, the first quantum dot binds to the first binding site of the biological target and the second quantum dot binds to the second binding site of the biological target to form a first quantum dot/biological target/second quantum dot complex; exposing the first quantum dot/biological target/second quantum dot complex to a single irradiation source emitting radiation at a single wavelength; and simultaneously detecting the emission energy from each of the first quantum dot and the second quantum dot, wherein simultaneously detecting the emission from the first quantum dot and the second quantum dot is correlated to the detection of the first quantum dot/biological target/second quantum dot complex.

Another exemplary method of detecting a biological target, among others, includes: providing a first bead and a second bead, wherein different wavelengths of emission energy are emitted from the first bead and the second bead, wherein the emission from the first bead is at a first wavelength and the emission from the second bead is at a second wavelength, wherein the first wavelength and the second wavelength are detectably distinguishable, wherein the first bead includes a first biomolecule having an affinity for a first binding site of a biological target, wherein the second bead includes a second biomolecule having an affinity for a second binding site of the biological target, and wherein the biological target includes the first binding site and the second binding site; mixing the first bead and the second bead with a sample, wherein if the sample includes the biological target, the first bead binds to the first binding site of the target molecule and the second bead binds to the second binding site of the target molecule to form a first bead/biological target/second bead complex; exposing the first bead/biological target/second bead complex to a single irradiation source emitting radiation at a single wavelength; and simultaneously detecting the emission energy emitted from each of the first bead and the second bead, and wherein simultaneously detecting the emission from the first bead and the second bead is correlated to the detection of the first bead /biological target/second bead complex.

One exemplary single-molecule detection system, among others, includes: a channel for flowing a fluid that includes a fluorescent/biological target complex, wherein the fluorescent/biological target complex emits at least two radiation energy signals upon irradiation by a single wavelength of radiation energy, wherein each radiation energy signal is at a distinguishably different wavelength; an irradiation and optic system including an irradiation source configured to direct a single wavelength of an irradiation beam onto a portion of the channel, wherein the irradiation and optic system is configured to direct the at least two emitted radiation energy signals from the fluorescent/biological target complex to the detector and co-incidence system; a detector and co-incidence system including at least two detectors, wherein each detector includes a filter that allows only a wavelength for one of the radiation energy signals emitted from the fluorescent/biological target complex to pass through each of the filters, wherein the detector and co-incidence system includes a co-incidence detector interfaced to one of the detectors and a delay generator interfaced to the other detector, wherein the delay generator and the co-incidence detector are directly interfaced, wherein the co-incidence detector is configured to determine if co-incident signals are emitted from the fluorescent/biological target complex; and an analysis system in communication with the co-incidence detector, wherein the analysis system receives data from the co-incidence detector if the detectors simultaneously receive radiation energy signals emitted from the fluorescent/biological target complex.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1(a) and 1(b) illustrate embodiments of the fluorescent/biological target complexes.

FIG. 2 illustrates a single-molecule detection system.

FIG. 3 illustrates a computer system that implements aspects of the single-molecule detection system.

FIG. 4 illustrates schematic diagrams illustrating the principles of single-molecule sandwich assays using color-coded nanopartcles. FIG. 4(a) illustrates simultaneous double-site binding for protein and nucleic acid detection; FIG. 4(b) illustrates free nanoparticle probes and bound sandwich pairs moving across a tightly focused laser beam; and FIG. 4(c) illustrates fluorescence emission spectra of color-coded quantum dots and energy-transfer nanoparticles. The left panel shows green and red QDs simultaneously excited with a single light source at 420 nm, and the right panel shows green and red energy-transfer nanoparticles excited at the same wavelength. The arrows indicate the relative position of the excitation laser wavelength (488 nm) used in single-molecule detection. Note that the red energy-transfer particle has a broader and less symmetric emission spectrum than that of red QD. The green curve in the right panel arises from a single green dye embedded in the nanoparticle, without resonance fluorescence energy transfer or FRET.

FIG. 5 illustrates a representative example of an instrumental diagram showing real-time detection of single nanoparticles and correlated sandwich pairs flowing in a small capillary. It should be noted that: LF-1=laser filter; DC-1=dichroic filter in the microscope filter cube; DC-2=dichroic filter outside the microscope side port; BP-1=bandpass filter, HQ514 M10; BP-2=bandpass filter D670 M40; AL-1 and AL-2=aspheric focusing lenses; ADP-1 and APD-2=single-photon counting avalanche photodiodes; MCS=multichannel scalar; and PC=personal computer.

FIG. 6 illustrates representative logic and electronic hardware for real-time correlation analysis of single photon events on two detectors (ADP1- and APD-2).

FIG. 7 illustrates real-time photon burst data obtained from DNA-conjugated nanoparticles in a microfluidic channel, and simultaneously recorded from APD-1 (green channel), APD-2 (red channel), and the co-incidence channel (yellow). FIG. 7(a) Green nanoparticle probes (SEQ. ID. NO: 1, 5′-CTTCAGTTTCTCGGG-(A)10-3′-nanoparticle, 7.2 pM); FIG. 4(b) Red nanoparticle probes (SEQ. ID. NO: 2, nanoparticle-5′-(A)10-CTCCTCCAGCTCCTT-3′, 4.2 pM): FIG. 7(c) a mixture of 7.2 pM green probes and 4.2 pM red probes (without a complementary DNA target); and FIG. 7(d) a mixture of 7.2 pM green probes and 4.2 pM red probes with a complementary DNA target (SEQ. ID. NO: 3,5′-CCCGAGAAACTGAAG-AAGGAGCTGGAGGAG-3′,4.0 pM). Boldfaced and underlined letters denote complementary sequences. Microchannel inner diameter=2.0 μm; liquid flow velocity=0.55 mm/s; data acquisition speed=1000 data points per second (integration time=1 ms); correlation time=3.5 μs; laser wavelength=488 nm; and laser intensity=100.0 μW.

FIG. 8(a) illustrates complex nuclear lysates, FIG. 8(b) illustrates using large energy-transfer particles of different sizes, and FIG. 8(c) illustrates using two-color semiconductor QDs. FIG. 8(a) illustrates an example including green and red nanoparticle probes mixed with Raji-B cell lysates with and without a complementary DNA target. The experimental conditions were the same as in FIG. 7, except that the target DNA concentration was 2 pM. FIG. 8(b) illustrates co-incidence detection data using 200-nm green probes (4.5 pM) and 100-nm red probes (3.0 pM) in the presence of a complementary target (2 pM). FIG. 8(c) illustrates co-incidence detection data using 3.5-nm green QD probes (8 pM) and 5.5-nm red QD probes (7 pM) in the presence of a complementary target (4 pM).

FIG. 9 illustrates real-time photon burst data obtained from antibody-conjugated nanoparticles in a microfluidic channel, and recorded simultaneously from APD-1 (green channel), APD-2 (red channel), and the co-incidence channel (yellow). FIG. 9(a) illustrates a mixture of 4.7 pM green probes and 4.7 pM red probes (conjugated to rat anti-monoclonal antibodies) in the absence of the target protein; FIG. 9(b) illustrates the same as in FIG. 9(a) but with the target protein (50 pg/ml recombinant murine TNF-α). The other experimental conditions were the same as in FIG. 7.

FIG. 10 illustrates real-time photon burst data obtained from antibody-conjugated nanoparticles mixed with intact RSV viruses, and recorded simultaneously from APD-1 (green channel), APD-2 (red channel), and the co-incidence channel (yellow). FIG. 10(a) illustrates mutated virus RSV ΔG (with the G protein deleted); and FIG. 10(b) illustrates wild-type RSV with both F and G proteins expressed on the virus surface. RSV (4×10⁶ pfu) was allowed to bind to two monoclonal antibodies against the G-attachment protein epitope (clone 131-2G) and the F-fusion protein epitope (clone 131-2A), respectively. The F-protein antibody was conjugated to red nanoparticles, and the G-protein antibody was conjugated to green nanoparticles. A 9-μm capillary channel was used to prevent clogging, and the other experimental conditions were the same as in FIG. 7.

FIG. 11 illustrates single-molecule counting efficiencies and standard errors at different target DNA concentrations. The actual target counts are shown in 10 data points (squares), and the theoretical numbers of target molecules passing through the probe volume is shown by a dotted line. The counting data are the mean values±standard error (n=10).

FIGS. 12(a) and 12(b) represent a schematic illustration of single virus detection and surface protein determination. A virus bound with complementary nanoparticle-antibody probes flows into a confocal volume (confined by blue lines) where fluorescent nanoparticles are excited by a laser. The photons from green and red fluorescent nanoparticles are separated (spectra at the bottom) and analyzed for time correlation (coincidence). FIG. 12(a) illustrates the virus that does not bind to both the probes (left) will not show coincidence signals (dotted lines), while the virus that binds to both the probes (right) will produce time-correlated photons from the red and the green detection channels. FIG. 12(b) illustrates that when a single-color probe is used, the virus that binds to several probes (virus 1) will exhibit greater intensity in the integrated photon count spectrum. The bottom graph shows the expected trend in average intensity calculated from several photon count spectra.

FIG. 13 illustrates time-correlated coincidence signals for virus detection in solution. 40 nm nanoparticles conjugated to RSV anti-F or anti-G protein monoclonal antibodies were used to produce red or green photon emission, respectively. PIV3, used as a control, produced low red or green photon counts and did not show coincidence signals FIG. 13(a), while coincident peaks were observed for the RSV/A2 FIG. 13(b). The time for detection was 8 seconds, and signals were acquired and analyzed in real time (without any delay).

FIG. 14 illustrates time-correlated coincidence signals for virus detection in solution. 40 nm nanoparticles conjugated to RSV anti-F or anti-G protein monoclonal antibodies were used to produce red or green photon emission, respectively. RSVΔG, used as a control, produced low green photon counts as expected and did not show coincidence signals FIG. 14(a), while coincident peaks were observed for RSV/A2 FIG. 13(b). The magnitude of red channel signal for RSVΔG was low compared to RSV/A2, suggestive of lower F protein expression compared to RSV/A2. The time for detection was 8 seconds, and signals were acquired and analyzed in real time (without any delay).

FIG. 15 illustrates the measurement of RSV F protein expression on virus particles. QDs coupled with anti-RSV F protein monoclonal antibody and incubated with different viruses generated different photon counts based on anti-F protein nanoparticle aggregation. No signal above the unconjugated QD control was observed for PIV3. Differences in signal intensities obtained between RSV/A2 and RSVΔG are suggestive of differences in the level of F protein expression.

FIG. 16 illustrates a virus protein expression determined by the average photon peak intensities. Photon counts were determined from 35 individual runs (8 seconds each) and averaged for each virus. The bars indicate the mean±standard deviations.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

In accordance with the present disclosure there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells and Enzymes” (IRL Press, (1986)); B. Perbal, “A Practical Guide To Molecular Cloning” (1984), each of which is incorporated herein by reference.

As used herein, the terms “antibody” and “antibodies” can include, but are not limited to, monoclonal antibodies, multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (e.g., anti-Id antibodies to antibodies of the disclosure), and epitope-binding fragments of any of the above. In particular, antibodies include immunoglobulin molecules and immunologically active fragments of immunoglobulin molecules (e.g., molecules that contain an antigen binding site). Immunoglobulin molecules can be of any type (e.g., IgG, IgE, IgM, IgD, IgA and IgY), class (e.g., IgG1, IgG2, IgG3, IgG4, IgA1 and IgA2), or subclass. The antibodies may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. The antibodies may be monospecific, bispecific, trispecific, or of greater multispecificity.

As used herein, “humanized” describes antibodies wherein some, most, or all of the amino acids outside the complementarity-determining regions (CDR regions) are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody would retain a similar antigenic specificity as the original antibody.

As used herein, “antigen” describes a compound, a composition, or a substance that can stimulate the production of antibodies or a T-cell response in a host.

The term “polypeptides” includes proteins and fragments thereof Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically, or metabolically modified forms of polynucleotides, as well as the chemical forms of DNA and RNA characteristic of viruses and cells, including simple and complex cells, inter alias.

“Aptamers” may be high affinity, high specificity polypeptide, RNA, or DNA-based probes produced by in vitro selection experiments. Aptamers may be generated from random sequences of nucleotides or amino acids, selectively screened by absorption to molecular antigens or cells, and enriched to purify specific high affinity binding ligands, for example. In solution, aptamers may be unstructured but may fold and enwrap target epitopes providing specific binding recognition. The unique folding of the nucleic acids around the epitope, for example, affords discriminatory intermolecular contacts through hydrogen bonding, electrostatic interaction, stacking, and shape complementarity.

Use of the phrase “biomolecule” is intended to encompass deoxyribonucleic acid (DNA), ribonucleic acid (RNA), nucleotides, oligonucleotides, nucleosides, polynucleotides, proteins, peptides, polypeptides, selenoproteins, antibodies, antigens, protein complexes, aptamers, combinations thereof, and the like.

Use of “biological” or “biological target” is intended to encompass biomolecules (e.g., deoxyribonucleic acid (DNA), ribonucleic acid (RNA), nucleotides, oligonucleotides, nucleosides, polynucleotides, proteins, peptides, polypeptides, selenoproteins, antibodies, antigens, protein complexes, aptamers, combinations thereof) and the like. In particular, biological or biological target can include, but is not limited to, naturally occurring substances such as polypeptides, polynucleotides, lipids, fatty acids, glycoproteins, carbohydrates, fatty acids, fatty esters, macromolecular polypeptide complexes, vitamins, co-factors, whole cells, eukaryotic cells, prokaryotic cells, micelles, microorganisms such as viruses, bacteria, protozoa, archaea, fungi, algae, spores, apicomplexan, trematodes, nematodes, mycoplasma, or combinations thereof. In addition, the biological target can include native intact cells, viruses, bacterium, and the like.

The term “virus” can include, but is not limited to, RNA and DNA viruses. In particular, the virus can include, but is not limited to, negative-sense and positive-sense RNA viruses and single stranded (ss) and double stranded (ds) DNA viruses. The ds group I DNA viruses include the following families: Adenoviridae, Herpesviridae, Papillomaviridae, Polyomaviridae, Poxviridae, and Rudiviridae. The group II ssDNA viruses include the following families: Microviridae, Geminiviridae, Circoviridae, Nanoviridae, and Parvoviridae. The ds group III RNA viruses include the following families: Birnaviridae and Reoviridae. The group IV postive-sense ssRNA virus familes: Arteriviridae, Coronaviridae, Astroviridae, Caliciviridae, Flaviviridae, Hepeviridae, Picornaviridae, and Togaviridae. The group V negative-sense ssRNA virus familes: Bornaviridae, Filoviridae, Paramyxoviridae, Rhabdoviridae, Arenaviridae, Bunyaviridae, and Orthomyxoviridae. The term “virus” includes one of a number of strands of the virus and/or a mutated version of a virus or of one of a number of strands of a virus. The term “virus” includes intact viruses as well.

The term “bacteria” can include, but is not limited to, Gram-positive or Gram-negative bacteria. The Gram-positive bacteria may include, but is not limited to, Gram positive Cocci (e.g., Streptococcus, Staphylococcus, and Enterococcus). The Gram-negative bacteria may include, but is not limited to, Gram negative rods (e.g., Bacteroidaceae, Enterobacteriaceae, Vibrionaceae, Pasteurellae and Pseudomonadaceae).

Use of the term “affinity” can include biological interactions and/or chemical interactions. The biological interactions can include, but are not limited to, bonding or hybridization among one or more biological functional groups located on the first biomolecule or biological target and the second biomolecule or biological target. In this regard, the first (or second) biomolecule can include one or more biological functional groups that selectively interact with one or more biological functional groups of the second (or first) biomolecule. The chemical interaction can include, but is not limited to, bonding among one or more functional groups (e.g., organic and/or inorganic functional groups) located on the biomolecules.

### Discussion

In accordance with the purpose(s) of the present disclosure, as embodied and broadly described herein, embodiments of the present disclosure, in one aspect, relate to methods of measuring and/or detecting biological targets (e.g., native viruses), methods of distinguishing among the same type of biological target (e.g., sub-species of the same virus), single-molecule detection systems, fluorescent/biological target complexes, methods of using fluorescent/biological target complexes, and the like. Embodiments of the present disclosure enable detection of single biological targets in real-time (e.g., seconds). In addition, embodiments of the present disclosure enable real-time detection of single native biological targets (e.g., native biomolecules and intact viruses) by simultaneously detecting co-incident emitted radiation from compounds or structures bound to the biological target. Embodiments of the present disclosure can be used in chemical analysis, molecule diagnostics, bioterrorism agent detection, molecular imaging, intracellular single-molecule imaging, studying ligand-receptor binding, and studying cell signaling.

The fluorescent/biological target complexes include, but are not limited to, a first fluorescent compound (or structure), a second fluorescent compound (or structure), and a biological target. In another embodiment, the fluorescent/biological target complexes include three or more fluorescent compounds. The fluorescent compounds can include, but is not limited to, quantum dots, dyes, fluorescence resonance energy transfer beads or FRET-beads (e.g., beads incorporating fluorescent compound donor/acceptor pairs), and dye-beads (e.g., beads incorporating a fluorescent compound dye). For reasons of clarity, portions of the disclosure will be discussed with respect to a dual fluorescent/biological target complex, but it is to be understood and envisioned that three or more fluorescent compounds can be used in embodiments of the present disclosure.

The biological target can include, but are not limited to, viruses, bacteria, cells, microorganisms, artificially constituted nanostructures (e.g., micelles), proteins, polypeptides, antibodies, antigens, aptamers (polypeptide and polynucleotide), polynucleotides, and the like, as well as those biological targets described in the definition section above. Embodiments of the present disclosure can be conducted without labeling the biological target. Thus, native, unamplified samples can be used. It should also be noted that the samples do not have to be treated and/or portions of the sample separated from other portions of the sample. For example, a pure blood sample can be used without additional preparation, separation, purification, and/or other treatment.

The biological target includes, but is not limited to, a first binding site or moiety and a second binding site or moiety, where the first binding site and the second binding site are not the same (e.g., in that they bind to different biological targets). In an embodiment, the binding sites (e.g., the first binding site and the second binding site) are an inherent part of the biological targets. The first binding site and the second binding site can each include, but are not limited to, a protein or a portion thereof, a polypeptide or a portion thereof, an antibody or a portion thereof, an antigen or a portion thereof, a polynucleotide or a portion thereof, and those biological targets described in the definition section above, of the biological target that have an affinity for the first binding molecule and the second binding molecule, respectively. In an embodiment, each of the binding sites or moieties can include, but is not limited to, a protein expressed on the surface of a virus.

The first fluorescent compound includes, but is not limited to, a first binding biomolecule having an affinity for the first binding site of the biological target. The second fluorescent compound includes, but is not limited to, a second binding biomolecule having an affinity for the second binding site of the biological target. The first binding biomolecule and the second binding biomolecule are not the same (e.g., in that they bind to different binding sites or moieties), and the first binding site and the second binding site are not the same (e.g., in that they bind to different biomolecules). For example, the first binding biomolecule does not bind or substantially bind to the second binding site, and the second binding biomolecule does not bind or substantially bind to the first binding site.

The first binding biomolecule and the second binding biomolecule can each include, but are not limited to, a protein or a portion thereof, a polypeptide or a portion thereof, an antibody or a portion thereof, an antigen or a portion thereof, an aptamer (polypeptide or polynucleotide), a polynucleotide or a portion thereof, and those biomolecules described in the definition section above, that have an affinity for the first binding site or the second binding site, respectively.

Each of the first fluorescent compound and the second fluorescent compound can be designed to include one first binding biomolecule or one second binding biomolecule, respectively. Designing the first fluorescent compound and the second fluorescent compound to have one or about one binding biomolecule ensures that the first fluorescent compound and the second fluorescent compound only bind to a single biological target. In another embodiment, the fluorescent compounds can include more than one binding biomolecule and alternative methods can be used to ensure that only appropriately labeled target molecules are included in the analysis. The binding biomolecules can be attached to the fluorescent compounds directly (e.g., the surface of the fluorescent compound) or indirectly (e.g., via a linker compound, polypeptide, polynucleotide, and the like or are attached to a bead that includes the fluorescent compound).

The first fluorescent compound and the second fluorescent compound emit radiation (e.g., characteristic of the type of fluorescent compound) at detectably distinguishable regions or distinguishably different regions of the spectrum (e.g., at different wavelengths that can be distinguished using spectral analysis or the like). In an embodiment, each of the first fluorescent compound and the second fluorescent compound (e.g., a pair of quantum dots or dyes) absorb radiation energy at the same wavelength, while emitting radiation at distinguishably different wavelengths than one another and distinguishably different than the wavelength of the radiation from the irradiation source.

As mentioned above, embodiments of the present disclosure include methods of measuring and/or detecting biological targets. In general, once the first fluorescent compound and the second fluorescent compound bind to the biological target, then the dual fluorescent/biological target complex can be detected. In general, an irradiation source (e.g., a laser system emitting a laser beam) irradiates the dual fluorescent/biological target complex at a single wavelength. In an embodiment where the first fluorescent compound and the second fluorescent compound are quantum dots, each quantum dot absorbs the irradiation energy and each emits characteristic radiation at a particular wavelength, where the wavelengths are detectably different or the wavelengths are distinguishably different. Co-incidence detection and/or measurement of the emission of each of the wavelengths is an indication that the biological target of interest is present. If only one of the emissions is measured, then the emission can not be correlated to the biological target. In another embodiment, one or both of the quantum dots can be replaced with a dye, where each dye absorbs radiation from the irradiation source at the same wavelength and emits radiation at distinguishably different wavelengths.

For example, a sample that includes biological targets is mixed with the first fluorescent compound and the second fluorescent compound. The first fluorescent compound and the second fluorescent compound are allowed to bind with the biological target to form the dual fluorescent/biological target complex. The solution can then be introduced to and flowed in a channel (e.g., microchannel or microcapillary tube), where the channel passes by the radiation emitted from the irradiation source. A single wavelength of radiation is emitted from the radiation source and passes through the channel and impinges upon the dual fluorescent/biological target complexes as they flow through the channel. The quantum dots emit characteristic radiation that is simultaneously detected by detectors (e.g., two photon-counting photodiodes), where one detector detects one characteristic wavelength and another detector detects the other characteristic wavelength (e.g., with the use of a filtering system). Co-incidence detection of both emissions of radiation indicates that the biological target is present in the sample. The biological targets are detected one at a time as they pass through the channel and through the radiation beam. Other molecules present will not emit radiation at both characteristic emission wavelengths. Real-time analysis of the data can distinguish emissions that are correlated to the biological targets and those emissions that are not correlated to the biomolecules (e.g., unbound quantum dots or molecules bound to only one quantum dot).

In another embodiment, the first fluorescent compound and the second fluorescent compound are part of a fluorescence resonance energy transfer (FRET) system. FRET is a radiationless process in which energy is transferred from an excited donor molecule to an acceptor molecule. The FRET system includes a first bead (first FRET-bead) that includes the donor and the acceptor (a first donor/acceptor pair (analogous to the first fluorescent compound)) in close proximity and a second bead (second FRET bead) that includes the donor and the acceptor (a second donor/acceptor pair (analogous to the second fluorescent compound)) in close proximity. In another embodiment, dye-beads (e.g., a first dye-bead and a second dye bead) can be used, where each bead includes a single type of dye that emits radiation at a characteristic wavelength.

In short, the donor molecule is a fluorophore that absorbs light energy at a characteristic wavelength. This characteristic wavelength is also known as the excitation wavelength. The energy absorbed by the donor molecule is subsequently released through various pathways, one being emission of photons to produce fluorescence at a characteristic wavelength. The characteristic wavelength of light being emitted is known as the emission wavelength and is an inherent characteristic of the donor molecule. Radiationless energy transfer is the quantum-mechanical process by which the energy of the excited state of one fluorophore (the donor molecule) is transferred without actual photon emission to a second fluorophore (the acceptor molecule). That energy may then be subsequently released at a characteristic emission wavelength of the acceptor molecule. The donor molecule has an excited state of higher energy than that of the acceptor molecule. The emission spectrum of the donor molecule overlaps with the excitation spectrum of the acceptor molecule. In addition, the donor molecule and the acceptor molecule should be sufficiently close proximity. The distance over which radiationless energy transfer is effective depends on many factors including the fluorescence quantum efficiency of the donor, the extinction coefficient of the acceptor, the degree of overlap of their respective spectra, the refractive index of the medium, and the relative orientation of the transition moments of the two fluorophores. The donor and the acceptor are proximally positioned in the FRET beads so that FRET can occur. The emission of the donor molecule and the acceptor molecule are detectably distinguishable. In embodiments including two or more beads, detection and/or measurement of two or more co-incidence (at least one emission from each bead) emissions of each of the wavelengths is an indication that the biological target of interest is present. Other molecules present will not emit radiation at the characteristic emission wavelengths. Analysis of the data can distinguish the emissions correlated to the biological targets and those emissions that are not correlated to the biomolecules (e.g., unbound FRET-beads or molecules bound to only one FRET-bead).

The bead (e.g., FRET-beads and dye-beads) can include, but is not limited to, a porous bead or a bead that, as it is being formed, incorporates the donor molecule and the acceptor molecule with the bead. An advantage of incorporating the acceptor and the donor in the bead is that the distance between the two molecules is not a limiting factor as in other FRET systems.

The beads (e.g., FRET-beads and dye-beads) can include, but are not limited to, silica beads and polymer beads (e.g., chromatographic beads), ceramic, and molecular sieves. The beads can be made of a material such as, but not limited to, a polymer, a co-polymer, a metal, a silica material, cellulose, ceramic, zeolite, and combinations thereof. In an embodiment, the beads are made of silica materials and polystyrene and polystyrene co-polymers (e.g., divinylbenzene, methacylic acid, maleic acid). It should be noted that the reference to “bead(s)” could refer to “FRET-bead(s)” or “dye-bead(s)”, and one can determine which is being described based on the context of the sentence or paragraph. In instances where “bead(s)” are referred to generally or generically, the description can be applied to either or both “FRET-bead(s)” or “dye-bead(s)” consistent with the descriptions provided herein.

The first bead includes, but is not limited to, the first binding biomolecule having an affinity for the first binding site of the biological target. The second bead includes, but is not limited to, a second binding biomolecule having an affinity for the second binding site of the biological target. The binding biomolecules are attached to the surface or sub-surface/sub-layer of the bead, rather than to the donor and/or acceptor molecules. In particular, the first binding biomolecule is attached (directly or indirectly) to the first bead, while the second binding biomolecule is attached (directly or indirectly) to the second bead.

The first bead includes a first donor/acceptor pair, while the second bead includes a second donor/acceptor pair. The first donor/acceptor pair and the second donor/acceptor pair emit radiation at distinguishably different wavelengths. Two or more signals can be detected to ensure that the first bead and the second bead are bound to the biological target. In another embodiment, three or more beads can be used to identify target molecules and appropriate donor/acceptor pairs can be selected.

For example, a sample that includes biological targets is mixed with the first bead and the second bead. The beads are allowed to bind with the biological target to form the dual fluorescent/biological target complex. The solution can then be introduced to and flowed in a channel (e.g., microchannel), where the channel passes by the radiation emitted from the irradiation source. A single wavelength of radiation is emitted from the radiation source and passes through the channel and impinges upon the fluorescent/biological target complexes as they flow through the channel. Each of the first donor molecule and the second donor molecule absorb the radiation from the radiation source and each emits a characteristic radiation at a characteristic wavelength. In addition, a radiationless energy transfer from the first donor molecule to the first acceptor molecule and from the second donor molecule to the second acceptor molecule occurs. Then the first acceptor molecule and the second acceptor molecule each emit radiation at a characteristic wavelength. The four emissions are at distinguishably different wavelengths. Two or more of the emitted radiation signals are simultaneously detected by detectors (e.g., two or more photon-counting photodiodes), where each detector detects at a different wavelength (e.g., with the use of a filtering system). Co-incident detection of two or more emissions of radiation (at least one from each bead) indicates that the biological target is present in the sample. The biological targets are detected one at a time as they pass through the channel and through the radiation beam. Other molecules present will not emit radiation at the characteristic emission wavelengths. Analysis of the data can determine the emissions correlated to the biological targets and those emissions that are not correlated to the biomolecules (e.g., unbound FRET-beads or molecules bound to only one FRET-bead).

In another embodiment, the biological target includes a plurality of first binding sites and/or a plurality of second binding sites. One or more first fluorescent compounds and/or the second fluorescent compounds (or one or more beads) can bind with the biological target. The relative amount of each of the first binding sites and the second binding sites can be determined based on the relative amounts of the emission of the first fluorescent compound and/or the second fluorescent compound. Embodiments of the present disclosure can be used to distinguish among different types of the same biological target (e.g., different strands of the same type of virus or the like). The same biological target may include two or more types, classes, subspecies, and the like, and each different type may have different amounts of the first binding site and the second binding site. The first binding biomolecule and the second binding biomolecule can be selected to have affinities for specific first binding sites and the second binding sites so that two or more biological targets can be distinguished based on the characteristic emission ratio of the first fluorescent compound and the second fluorescent compound (or donor/acceptor pairs).

The fluorescent compounds (e.g., the first fluorescent compound and the second fluorescent compound or one or more beads including the donor/acceptor pairs or dyes), the binding biomolecules (e.g. the first binding biomolecule and/or second biomolecule), the binding sites, the emission of the fluorescent compounds or donor/acceptor pairs, and the like, can be designed and/or tailored to detect, measure, quantify, and/or quantitate, the presence of the one or more target molecules. In addition, embodiments of the present disclosure can be designed and/or tailored to differentiate between or among different subspecies of the same genus of the biological target.

In an illustrative embodiment, the biological target is a virus. In another illustrative embodiment, the virus is a RSV virus, and the binding biomolecules are selected to distinguish among different types or subspecies of RSV viruses. In another embodiment, multiple target molecules (e.g., different genus of a virus and/or different species or subspecies of the same genus) may be present in a sample, and multiple pairs of fluorescent compounds or beads are used to detect the different types of target molecules present.

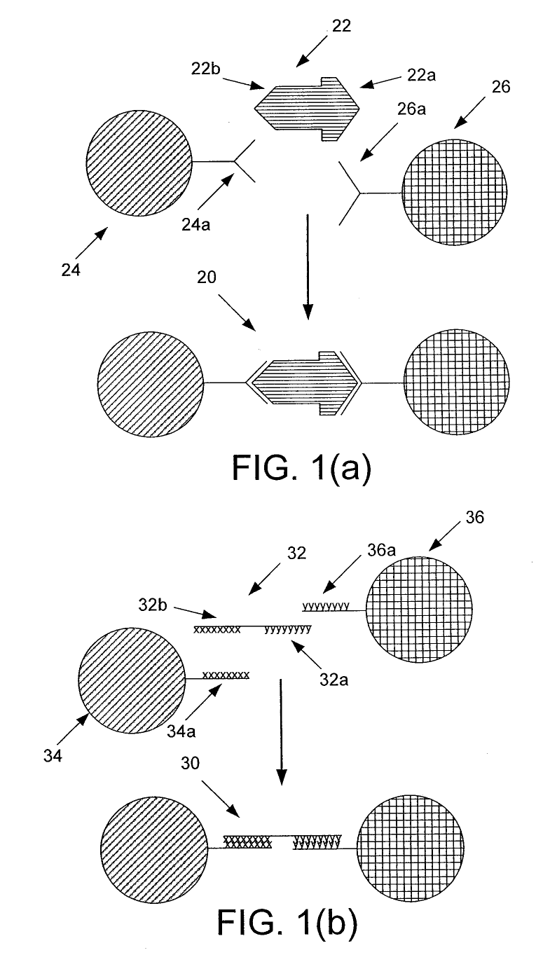

FIGS. 1(a) and 1(b) illustrate embodiments of the fluorescent/biological target complexes 20 and 30, respectively. FIG. 1(a) illustrates a biological target 22, a first fluorescent compound 24, and a second fluorescent compound 26. The biological target 22 includes, but is not limited to, a first binding site 22a and a second binding site 22b. The first fluorescent compound 24 includes, but is not limited to, binding biomolecule polypeptide 24a having an affinity for the second binding site 22b. The second fluorescent compound 26 includes, but is not limited to, binding biomolecule polypeptide 26a having an affinity for the first binding site 22a. The first fluorescent compound 24 and the second fluorescent compound 26 can each be a quantum dot or a FRET-bead (each bead including a donor/acceptor pair). In other embodiments, the first fluorescent compound 24 and the second fluorescent compound 26 can each be a dye-bead or a dye.

FIG. 1(b) illustrates a biological target polynucleotide 32, a first fluorescent compound 34, and a second fluorescent compound 36. The biological target polynucleotide 32 includes, but is not limited to, a first binding site 32a and a second binding site 32b. The first fluorescent compound 34 includes, but is not limited to, a binding biomolecule polynucleotide 34a having an affinity for the second binding site 32b. The second fluorescent compound 36 includes, but is not limited to, a binding biomolecule polynucleotide 36a having an affinity for the first binding site 32a. The first fluorescent compound 34 and the second fluorescent compound 36 can each be a quantum dot or a FRET-bead (each bead including a donor/acceptor pair). In other embodiments, the first fluorescent compound 34 and the second fluorescent compound 36 can each be a dye-bead or a dye.

### Single-Molecule Detection System

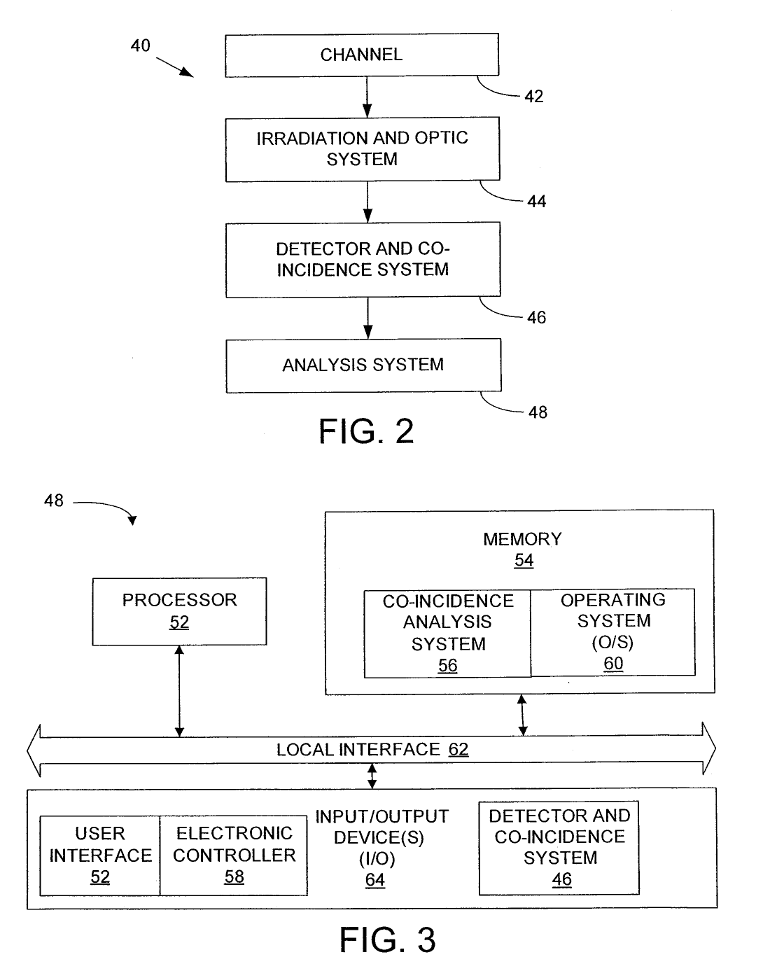

The sample including the fluorescent/biological target (e.g., dual fluorescent/biological target) can be analyzed using a single-molecule detection system 40 as shown in FIG. 2. The single-molecule detection system 40 provides for real-time (e.g., within seconds) results of the presence of biological targets in a sample. The real-time correlation analysis of single photon events allows rapid detection of biological targets in a few seconds. The real-time results are advantageous since other techniques take much longer to provide results (e.g., 1 hour to days). In addition, the single-molecule detection system 40 used in conjunction with fluorescent compounds or beads to form fluorescent/biological targets (e.g., dual fluorescent/biological target complexes) enables a single irradiation source emitting at a single wavelength to determine the presence of the biological target, which removes problems with par-focality and reduces false-positives. The single-molecule detection system 40 is less expensive since only a single irradiation source is used. The single-molecule detection system 40 is also technically less complex since two or more lasers do not need to be focused on the same area. The single-molecule detection system 40 is precise and reproducible when counting about 10 or more biological targets.

The single-molecule detection system 40 includes, but is not limited to, a channel 42 (e.g., a microchannel or microcapillary channel), an irradiation and optic system 44, a detector and co-incidence system 46, and an analysis system 48.

The channel 42 functions to flow a sample from one end of the channel 42 to another end of the channel 42. The channel 42 can have a length of about 1 μm to an appropriate length (e.g., sub-centimeter to hundreds of centimeters) for the setup and a width of about 250 nm and above. The length and/or the width can be modified for particular implementations of the single-molecule detection system 40. The channel 42 is configured to receive a sample and to flow the sample through the irradiation and optic system 44.

The irradiation and optic system 44 functions to generate and direct radiation generated by the irradiation and optic system 44 as well as to direct radiation emitted by components (e.g., dual fluorescent/biological target complexes) under analysis. The irradiation and optic system 44 includes, but is not limited to, an irradiation source (e.g., a laser source) and an optic system. The irradiation source emits a radiation beam at a single wavelength that passes through a portion (a known volume) of the channel 42. If a fluorescent compound, a dual fluorescent/biological target complex, and/or a complex having only a single fluorescent compound attached thereto, passes through the radiation beam, one or more radiation emissions may be generated. The optic system is configured to direct the radiation beam through a portion (e.g., a know volume dependent at least upon the radiation beam width and dimensions of the channel 42) of the channel. In addition, the optic system is configured to direct emitted radiation from one or more components present in the channel 42 at a given time to the detector and co-incidence system 46.

The detector and co-incidence system 46 functions to detect radiation from the components under analysis and/or to determine if radiation signals are received simultaneously for co-incidence detection. The detector and co-incidence system 46 includes, but is not limited to, two or more detectors (e.g., photon detectors (e.g., photodiodes, photomultipliers, and CCDs)) and a co-incidence detection system. The irradiation and optic system 44 includes wavelength filters to ensure only certain wavelengths are detected by the detectors. For example, a first filter only allowing a certain wavelength (e.g., the characteristic wavelength of the first fluorescent compound, the first donor, or the first acceptor) to pass through the first filter can be used for one detector, while a second filter only allowing a certain wavelength (e.g., the characteristic wavelength of the second fluorescent compound, the second donor, or the second acceptor) to pass through the second filter (different than that of the first filter) can be used for another detector. Filters are well known in the art and one skilled in the art can select appropriate filters for the single-molecule detection system 40. Therefore, two detectors can detect simultaneously emitted signals at different wavelengths without one signal interfering with the other signal.

The detector and co-incidence system 46 can include, but is not limited to, a co-incidence detector interfaced to one of the detectors and a delay generator interfaced to the another detector, where the delay generator and the co-incidence detector are interfaced. Under appropriate conditions, data from the detector and co-incidence system 46 is relayed to the analysis system 48 and analyzed by the co-incidence analysis system 56 of FIG. 3. For example, if a signal is generated by the first detector, then the co-incidence detector can determine if a signal is generated by the second detector by communicating with the delay generator. Data (e.g., time and the number of signals) regarding both signals can be relayed to the analysis system 48. The data can be reviewed in real-time.

It should be noted that the detector and co-incidence system 46 and the analysis system 48 could be hardware and/or software. Functions of the detector and co-incidence system 46 and the analysis system 48 can be combined into a single system or into three or more systems. The exact configuration of each of the detector and co-incidence system 46 and the analysis system 48 can be different depending on the particular set-up employed. Examples 1 and 2 describe illustrative embodiments of the single-molecule detection system 40.

As used herein, the term “co-incidence analysis system 56” indicates any system or program that can be implemented in software (e.g., firmware), hardware, or a combination thereof to perform the desired functions as set forth in this disclosure. In one example, the co-incidence analysis system 56 is implemented in software, as an executable program, and is executed by a special or general purpose digital computer, such as a personal computer (PC; IBM-compatible, Apple-compatible, or otherwise), workstation, minicomputer, or mainframe computer which may be adapted to interface with other parts of the device of the present disclosure, including, but not limited to, the detection and co-incidence system 46. An example of a general purpose computer that can implement the co-incidence analysis system 56 of the present disclosure is shown in FIG. 3.

Generally, in terms of hardware architecture, as shown in FIG. 3, the computer system includes a processor 52, memory 54, and one or more input and/or output (I/O) devices 64 (or peripherals) that are communicatively coupled via a local interface 62. The local interface 62 can be, for example but not limited to, one or more buses or other wired or wireless connections, as is known in the art. The local interface 62 may have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 52 is a hardware device for executing software, particularly that stored in memory 54. The processor 52 can be any custom made or commercially available processor, a central processing unit (CPU), an auxiliary processor among several processors associated with the computer of the analysis system 48, a semiconductor based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80x86 or Pentium series microprocessor from Intel Corporation, a PowerPC microprocessor from IBM, a Sparc microprocessor from Sun Microsystems, Inc, or a 68xxx series microprocessor from Motorola Corporation.

The memory 54 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 54 may incorporate electronic, magnetic, optical, and/or other types of storage media. Note that the memory 54 can have a distributed architecture, where various components are situated remote from one another, but can be accessed by the processor 52.

The software in memory 54 may include one or more separate programs, each of which comprises an ordered listing of executable instructions for implementing logical functions. In the example of FIG. 3, the software in the memory 14 includes the co-incidence analysis system 56 in accordance with the present disclosure and a suitable operating system (O/S) 60. A nonexhaustive list of examples of suitable commercially available operating systems 60 is as follows: (a) a Windows operating system available from Microsoft Corporation; (b) a Netware operating system available from Novell, Inc.; (c) a Macintosh operating system available from Apple Computer, Inc.; (e) a UNIX operating system, which is available for purchase from many vendors, such as the Hewlett-Packard Company, Sun Microsystems, Inc., and AT&T Corporation; (d) a LINUX operating system, which is freeware that is readily available on the Internet; (e) a run time Vxworks operating system from WindRiver Systems, Inc.; or (f) an appliance-based operating system, such as that implemented in handheld computers or personal data assistants (PDAs) (e.g., PalmOS available from Palm Computing, Inc., and Windows CE available from Microsoft Corporation). The operating system 60 essentially controls the execution of other computer programs, such as the co-incidence analysis system 56, and provides scheduling, input-output control, file and data management, memory management, and communication control and related services.

The co-incidence analysis system 56 is a source program, executable program (object code), script, or any other entity comprising a set of instructions to be performed. When a source program, then the program needs to be translated via a compiler, assembler, interpreter, or the like, which may or may not be included within the memory 54, so as to operate properly in connection with the O/S 60. Furthermore, the co-incidence analysis system 56 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, Matlab, IDL, and Ada.

The I/O devices 64 may include input devices, for example but not limited to, a keyboard, mouse, scanner, microphone, etc. Furthermore, the I/O devices 64 may also include output devices, for example but not limited to, a printer, display, etc. Finally, the I/O devices 64 may further include devices that communicate both inputs and outputs, for instance but not limited to, a modulator/demodulator (modem; for accessing another device, system, or network), a radio frequency (RF) or other transceiver, a telephonic interface, a bridge, a router, sensors, etc. In particular the I/O devices 64 may include a user interface 52 (which may include I/O devices such as a keyboard, mouse, display, etc.), and detector co-incidence system 46, and an electronic controller 58 to control irradiation and optic system.

If the computer is a PC, workstation, or the like, the software in the memory 54 may further include a basic input output system (BIOS) (omitted for simplicity). The BIOS is a set of essential software routines that initialize and test hardware at startup, start the O/S 60, and support the transfer of data among the hardware devices. The BIOS is stored in ROM so that the BIOS can be executed when the computer is activated.

When the computer is in operation, the processor 52 is configured to execute software stored within the memory 54, to communicate data to and from the memory 54, and to generally control operations of the computer pursuant to the software. The co-incidence analysis system 56 and the O/S 60, in whole or in part, but typically the latter, are read by the processor 52, perhaps buffered within the processor 52, and then executed.

When the co-incidence analysis system 56 is implemented in software, as is shown in FIG. 3, it should be noted that the co-incidence analysis system 56 can be stored on any computer readable medium for use by or in connection with any computer related system or method. In the context of this document, a computer readable medium is an electronic, magnetic, optical, or other physical device or system that can contain or store a computer program for use by or in connection with a computer related system or method. The co-incidence analysis system 56 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

In an alternative embodiment, where the co-incidence analysis system 56 is implemented in hardware, the co-incidence analysis system can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The co-incidence analysis system 56 can be implemented, in one embodiment, as a single module or, in some embodiments, as a distributed network of modules, where one or more of the modules can be accessed by one or more applications or programs or components thereof.

In an embodiment, the electronic controller system and any necessary components of the computer system described above may be implemented in hardware and miniaturized with any necessary solid state electronics for integration into a portable device.

The co-incidence analysis system 56 receives data from the detector and co-incidence system 46. As mentioned above, the data corresponds to signals from a fluorescent compound, a dual fluorescent/biological target complex, and/or a complex having only a single fluorescent compound attached thereto. The detector and co-incidence system 46 collects and relays data to the co-incidence analysis system 56 related to whether or not one or more signals are detected by two or more detectors, where the signals can be detected simultaneously. Each detector detects a signal at a specific wavelength. In other words, each detector may detect a signal at the same time, but at distinguishably different wavelengths.

In an embodiment, the co-incidence analysis system 56 receives data related to whether or not a signal was received at a first detector at a time T1 and to whether or not a signal was received at a second detector at a time T1. The signals can be integrated over a time T2 and displayed to the user. If a signal is received at the first detector and the second detector at a time T1, then a dual fluorescent/biological target complex was detected. Detection of only one signal from either the first detector or the second detector does not correspond to the detection of a dual fluorescent/biological target complex, but rather an unconjugated fluorescent compound (or bead) and/or a biological target having only a single fluorescent compound attached thereto. It should be noted that additional data analysis could be used to reduce false positives. Also, additional details regarding an illustrative embodiment are described in Examples 1 and 2.

It should be noted that an advantage of using quantum dots and FRET-beads (also know as “energy transfer beads” or “dye-doped beads”) eliminates the issue with parfocality. The elimination of parfocality reduces false positives and improves effective probe volume from 30% to about 100%, which achieves better throughput (e.g., more sample can be processed in shorter time and fewer target molecules passing through the probe volume are missed).

It should be noted that only a single irradiation source is needed when using embodiments of the present disclosure, which is in contrast to other systems that use two lasers. This reduces the instrumentation cost significantly as well as other technical issues related to using two lasers to produce simultaneous emissions.

Embodiments of the present disclosure provide an online co-incidence analysis to detect biological targets. This is a real time readout method that can allow multiplexed single biological target detection with embodiments of the present disclosure.

It should noted that quantum dots are much brighter than other molecules, which means that characteristics (e.g., protein expression levels) of the biological targets (e.g., viruses) can be measured more accurately using quantum dots.

It should be noted that embodiments of the present disclosure use a microfluidic capillary. Using the microfluidic capillary increases throughput of sample compared with other similar single molecule detection systems. Therefore, more sensitive detection can be done in a shorter amount of time.

It should be noted that a traditional fluorescence correlation spectroscopy system detects targets based on their diffusion characteristics or their mass. This limits the number of targets that can be detected simultaneously. Use of color coded quantum dots and FRET-beads overcomes this limitation and allows multiplexing.

### Quantum Dots

Quantum dots can include, but are not limited to, luminescent semiconductor quantum dots. In general, quantum dots include a core and a cap, however, uncapped quantum dots can be used as well. The “core” is a nanometer-sized semiconductor. While any core of the IIA-VIA, IIIA-VA, or IVA-IVA, IVA-VIA semiconductors can be used in the context of the present disclosure, the core is such that, upon combination with a cap, a luminescent quantum dot results. A IIA-VIA semiconductor is a compound that contains at least one element from Group IIA and at least one element from Group VIA of the periodic table, and so on. The core can include two or more elements. In one embodiment, the core is a IIA-VIA, IIIA-VA, or IVA-IVA semiconductor that ranges in size from about 1 nm to about 40 nm, about 1 nm to 30, about 1 nm to 20 nm, and about 1 nm to 10 nm. In another embodiment, the core is more preferably a IIA-VIA semiconductor and ranges in size from about 2 nm to about 10 nm. For example, the core can be CdS, CdSe, CdTe, ZnSe, ZnS, PbS, PbSe, or an alloy.

The “cap” is a semiconductor that differs from the semiconductor of the core and binds to the core, thereby forming a surface layer on the core. The cap typically passivates the core by having a higher band gap than the core. In one embodiment, the cap is a IIA-VIA semiconductor of high band gap. For example, the cap can be ZnS or CdS. Combinations of the core and cap can include, but are not limited to, the cap is ZnS when the core is CdSe or CdS, and the cap is CdS when the core is CdSe. Other exemplary quantum does include, but are not limited to, CdS, ZnSe, CdSe, CdTe, CdSeₓTe₁₋ₓ, InAs, InP, PbTe, PbSe, PbS, HgS, HgSe, HgTe, CdHgTe, and GaAs. The size of the cap is from about 0.1 to 10 nm, about 0.1 to 5 nm, and about 0.1 to 2 nm. Other illustrative quantum dots include, but are not limited to, a quantum dot having an emission at about 525 nm, a quantum dot having an emission at about 585 nm, a quantum dot having an emission at about 605 nm, a quantum dot having an emission at about 655 nm, and a quantum dot having an emission at about 685 nm.

The wavelength emitted (e.g., color) by the quantum dots can be selected according to the physical properties of the quantum dots, such as the size and the material of the nanocrystal. Quantum dots are known to emit light from about 300 nanometers (nm) to 2000 nm (e.g., UV, near IR, and IR). The colors of the quantum dots include, but are not limited to, red, blue, green, and combinations thereof. The color or the fluorescence emission wavelength can be tuned continuously. The wavelength band of light emitted by the quantum dot is determined by either the size of the core or the size of the core and cap, depending on the materials that make up the core and cap. The emission wavelength band can be tuned by varying the composition and the size of the QD and/or adding one or more caps around the core in the form of concentric shells.

The synthesis of quantum dots is well known and is described in U.S. Pat. Nos. 5,906,670; 5,888,885; 5,229,320; 5,482,890; 6,468,808; 6,306,736; 6,225,198, etc., International Patent Application WO 03/003015, (all of which are incorporated herein by reference) and in many research articles. The wavelengths emitted by quantum dots and other physical and chemical characteristics have been described in U.S. Pat. No. 6,468,808 and International Patent Application WO 03/003015 and will not be described in any further detail. In addition, methods of preparation of quantum dots are described in U.S. Pat. No. 6,468,808 and International Patent Application WO 03/003015 and will not be described any further detail.

In an embodiment, the quantum dot can be substantially coated or coated with a polymer or another compound. The polymer can include, but is not limited to, a polyamine, a capping ligand, a hydrophobic polymer layer, hydrophillic polymer layer, amphiphillic polymer layer, di- and/or tri-block copolymer layer, and combinations thereof. The coverage of the quantum dot with the polymer can range from about 1 to 100%. The thickness of the polymer layer can range from about 0.5 to 50 nm.

In another embodiment, the quantum dot can be capped with a capping ligand, which forms a layer on the quantum dot, and have a polymer layer disposed on the capping ligand. The capping ligand can include compounds such as, but not limited to, an O═PR₃ compound, an O═PHR₂ compound, an O═PHR₁ compound, a H₂NR compound, a HNR₂ compound, a NR₃ compound, a HSR compound, a SR₂ compound, and combinations thereof “R” can be a C₁ to C₁₈ hydrocarbon such as, but not limited to, linear hydrocarbons, branched hydrocarbons, cyclic hydrocarbons, substituted hydrocarbons (e.g., halogenated), saturated hydrocarbons, unsaturated hydrocarbons, and combinations thereof Preferably, the hydrocarbon is a saturated linear C₄ to C₁₈ hydrocarbon, a saturated linear C₆ to C₁₈ hydrocarbon, and a saturated linear C₁₈ hydrocarbon. A combination of R groups can be attached to P, N, or S. In particular, the capping ligand can be selected from, but is not limited to, tri-octylphosphine oxide, stearic acid, and octyldecyl amine, oleic acid, and derivatives thereof.

A protein, an antibody, a polynucleotide, a polypeptide, an aptamer, a linker, and/or other compound can be attached directly to the quantum dot and/or attached to the polymer layer disposed on the quantum dot. In addition, the protein, the antibody, the polynucleotide, the polypeptide, the linker, and/or the other compound can be attached indirectly to the quantum dot and/or attached to the polymer layer disposed on the quantum dot. For example, the protein, the antibody, the polynucleotide, the polypeptide, and/or the other compound can be attached in series via one or more linkers.

In still another embodiment, the quantum dot can have a ligand coating layer (original ligands). For different types of compound depositions, the original ligands can be kept, partially replaced, or completely replaced by other types of ligands. The original and/or new ligands can include small molecules (e.g., trioctyl phosphine oxide, hexadecylamine, hexadecene, mercaptoacetic acid, oleic acid, citric acid, and derivatives thereof) or polymers (e.g., polymers, copolymers, surfactants, lipid, or derivatives thereof), which can adsorb quantum dots and also provide a linkage for the protein, the antibody, the polynucleotide, the polypeptide, the linker, and/or the other compound. The ligands and the quantum dot can interact (directly or indirectly) through interactions such as, but not limited to, covalent bonds, hydrophobic interactions, hydrophilic interactions, or pi-stacking, etc., depending on the surface of nanospecies and the molecular structure of ligands.

In another embodiment, the quantum dot can be overcoated with another polymer on top of their original ligands, through interactions such as, but not limited to, hydrophobic interactions, hydrophilic interactions, covalent bonding, etc, then the compound is adsorbed onto the polymer via chelating, hydrophobic interactions, hydrophilic interactions, pi-stacking, covalent bonding, combinations thereof, and the like.

Exemplary polymers for the overcoat can include those described above. For example, the overcoat polymer can include, but is not limited to, amphiphilic polymers, detergent and/or a lipid structure including detergent derivatives and lipid derivatives. The amphiphilic polymer can include, but is not limited to hydrocarbons and DTPA modified poly(acrylic acid), poly(maleic acid), poly(maleic anhydride), and the like. The detergents can include, but are not limited to, AOT, brij family, lgepal family, triton family, SDS, or derivatives of each. In particular, the detergents can include, dioctyl sulfosuccinate sodium salt, polyethylene glycol dodecyl ether, (octylphenoxy)polyethoxyethanol, octylphenyl-polyethylene glycol, t-octylphenoxypolyethoxyethanol, polyethylene glycol tert-octylphenyl ether, 4-(1,1,3,3-tetramethylbutyl)phenyl-polyethylene glycol, dodecyl sulfate sodium salt, and glycolic acid ethoxylate octyl ether. Further, the block copolymer can include lipids such as, but not limited to, lipid-PEG, natural lipids, synthetic lipids, sphingolipids, and derivatives of each.

The thickness of each layer disposed on the quantum dot can vary significantly depending on the particular application. In general, the thickness is about 0.5 to 20 nm, about 0.5 to 15 nm, about 0.5 to 10 nm, and about 0.5 to 5 nm.

As mentioned above, the protein, the antibody, the polynucleotide, the polypeptide, the aptamer, the linker, and/or other compounds, can be linked to the quantum dot using any stable physical and/or chemical association to the nanospecies directly or indirectly by any suitable means. For example, the component can be linked to the quantum dot using a covalent link, a non-covalent link, an ionic link, and a chelated link, as well as being absorbed or adsorbed onto the quantum dot. In addition, the component can be linked to the quantum dot through hydrophobic interactions, hydrophilic interactions, charge-charge interactions, it-stacking interactions, combinations thereof, and like interactions.

The linker can include a functional group (e.g., an amine group) on the layer disposed on the quantum dot and/or the linker can include a separate compound attached to the quantum dot or the layer at one end and the protein, the antibody, the polynucleotide, the polypeptide, the aptamer, the linker, other compounds, or another linker at the other end. The linker can include functional groups such as, but not limited to, amines, carboxylic acids, hydroxyls, thios, and combinations thereof. The linker can include compounds such as, but not limited to, DTPA, EDTA, DOPA, EGTA, NTA, and combinations thereof. In an embodiment, the linker and the chelator compound are the same, but in other embodiments they can be different. The percentage of linkers attached to the chelator compound, contrast agent, and/or anther linker can be about 0.1 to about 100%.

### Fluorescence Resonance Energy Transfer Compounds and Dyes

Fluorescence resonance energy transfer (FRET) is a radiationless process in which energy is transferred from an excited donor molecule to an acceptor molecule. The efficiency of this transfer is dependent upon the distance between the donor an acceptor molecules. Since the rate of energy transfer is inversely proportional to the sixth power of the distance between the donor and acceptor, the energy transfer efficiency is extremely sensitive to distance changes. Energy transfer is said to occur with detectable efficiency in the 1-10 nm distance range, but is typically 4-6 nm for favorable pairs of donor and acceptor.

Radiationless energy transfer is based on the biophysical properties of fluorophores. These principles are reviewed elsewhere (Lakowicz, 1983, Principles of Fluorescence Spectroscopy, Plenum Press, New York; Jovin and Jovin, 1989, Cell Structure and Function by Microspectrofluorometry, eds. E. Kohen and J. G. Hirschberg, Academic Press, both of which are incorporated herein by reference). Briefly, a fluorophore absorbs light energy at a characteristic wavelength. This wavelength is also known as the excitation wavelength. The energy absorbed by a flurochrome is subsequently released through various pathways, one being emission of photons to produce fluorescence. The wavelength of light being emitted is known as the emission wavelength and is an inherent characteristic of a particular fluorophore. Radiationless energy transfer is the quantum-mechanical process by which the energy of the excited state of one fluorophore is transferred without actual photon emission to a second fluorophore. That energy may then be subsequently released at the emission wavelength of the second fluorophore. The first fluorophore is generally termed the donor (D) and has an excited state of higher energy than that of the second fluorophore, termed the acceptor (A). The emission specturm of the donor overlap with the excitation spectrum of the acceptor, and that is why the donor and acceptor should be sufficiently close. The distance over which radiationless energy transfer is effective depends on many factors including the fluorescence quantum efficiency of the donor, the extinction coefficient of the acceptor, the degree of overlap of their respective spectra, the refractive index of the medium, and the relative orientation of the transition moments of the two fluorophores. In addition to having an optimum emission range overlapping the excitation wavelength of the other fluorophore, the distance between the donor and acceptor are sufficiently small to allow the radiationless transfer of energy between the fluorophores.

In FRET, for example, a donor is allowed to coexist with an acceptor in an appropriately designed system. Thus, FRET is allowed to take place between both molecules, so as to visualize the interaction between the first and second molecules.

Long-wavelength fluorescence from the other dye (an acceptor molecule) is also measured. The interaction between the molecules is visualized by using the difference between the amounts that both emitted. Only when both dyes are adjacent to each other due to the interaction of the two types of molecules, a decrease in the fluorescence of the donor molecule and an increase in the fluorescence of the acceptor molecule are observed.

The donor and acceptor fluorophores are selected so that the emission wavelength spectrum of the donor is within the excitation wavelength spectrum of the acceptor. An appropriate combination of donor and acceptor fluorophores should be selected. As used herein with regard to fluorescent labels, the term “appropriate combination” refers to a choice of reporter labels such that the emission wavelength spectrum of the donor moiety is within the excitation wavelength spectrum of the “acceptor” moiety.

The donor and acceptor molecules are incorporated into a FRET-bead. The distance between the donor and acceptor molecules is within the limits for FRET to occur. FRET-beads can include, but are not limited to, Fluospheres and Transfluospheres available from Invitrogen Inc.

The dyes can be incorporated in dye-beads. The dye-beads can include, but are not limited to, Fluospheres and Transfluospheres, available from Invitrogen Inc. As discussed above, the dyes are selected to emit radiation at distinguishably different wavelengths.

It should also be noted that the fluorescent compounds and dyes could be selected from the following list. Appropriate combinations of fluorescent compounds can be selected from fluorophores such as, but not limited to, fluorescein, Pyrene, Coumarin, BODIPY, Oregon green, Rhodamine, Alexa, Oyster and Texas Red, or cyanine dyes. The fluorescent compounds can include, but are not limited to: 1,5 IAEDANS; 1,8-ANS; 4-Methylumbelliferone; 5-carboxy-2,7-dichlorofluorescein; 5-carboxyfluorescein (5-FAM); 5-Carboxynapthofluorescein; 5-carboxytetramethylrhodamine (5-TAMRA); 5-FAM (5-carboxyfluorescein); 5-HAT (hydroxy tryptamine); 5-hydroxy tryptamine (HAT); 5-ROX (carboxy-X-rhodamine); 5-TAMRA (5-carboxytetramethylrhodamine); 6-carboxyrhodamine 6G; 6-CR 6G; 6-JOE; 7-amino-4-methylcoumarin; 7-aminoactinomycin D (7-AAD); 7-hydroxy-4-methylcoumarin; 9-amino-6-chloro-2-methoxyacridine; ABQ; Acid Fuchsin; ACMA (9-amino-6-chloro-2-methoxyacridine); Acridine Orange; Acridine Red; Acridine Yellow; Acriflavin; Acriflavin Feulgen SITSA; Aequorin (Photoprotein); AFPs—AutoFluorescent Protein—(Quantum Biotechnologies); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; chlorophyll; chromomycin A; chromomycin A; CL-NERF; CMFDA; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′ DCFDA; DCFH (dichlorodihydrofluorescein diacetate); DDAO; DHR (dihydrohodamine 123); Di-4-ANEPPS; Di-8-ANEPPS (non-ratio); DiA (4-Di-16-ASP); dichlorodihydrofiuorescein Diacetate (DCFH); DiD—Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; dopamine; DTAF; DY-630-NHS; DY-635-NHS; ELF 97; Eosin; Erythrosin; Erythrosin ITC; ethidium bromide; ethidium homodimer-1 (EthD-1); Euchrysin; EukoLight; Europium (III) chloride; EYFP; Fast Blue; FDA; Feulgen (Pararosaniline); FIF (Formaldehyde-Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine ; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); S65A; S65C; S65L; S65T; SBFI; Serotonin; Sevron Brilliant Red 2B; Sevron Brilliant Red 4G; Sevron Brilliant Red B; Sevron Orange; Sevron Yellow L; SITS; SITS (Primuline); SITS (stilbene isothiosulphonic acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; WW 781; X-Rhodamine; XRITC; Xylene Orange; Y66F; Y66H; Y66W; YO-PRO-1; YO-PRO-3; YOYO-1;YOYO-3, Sybr Green, Thiazole orange (interchelating dyes), or combinations thereof. Appropriate combinations of donor/acceptor pairs (e.g., absorption and emission wavelengths) or dyes can be incorporated into FRET-beads or dye beads, respectively.

Embodiments of the present disclosure include, but are not limited to, one or more of the following combinations: a Fluosphere-Transfluosphere, a Transfluosphere-Transfluosphere, a QD-QD, a Fluosphere- QD, a Transfluosphere-QD, a Dye-dye, a dye-QD, and a dye-Transfluosphere.

As discussed above in reference to quantum dots, the protein, the antibody, the polynucleotide, the polypeptide, the aptamer, the linker, and/or other compounds, can be linked to the beads (e.g., FRET-beads and/or dye-beads) using any stable physical and/or chemical association to the nanospecies directly or indirectly by any suitable means. For example, the component can be linked to the bead using a covalent link, a non-covalent link, an ionic link, and a chelated link, as well as being absorbed or adsorbed onto the quantum dot. In addition, the component can be linked to the quantum dot through hydrophobic interactions, hydrophilic interactions, charge-charge interactions, it-stacking interactions, combinations thereof, and like interactions.

The linker can include a compound or functional group attached to the bead at one end and attached to the protein, the antibody, the polynucleotide, the polypeptide, the aptamer, the linker, other compounds, or another linker at the other end. The linker can include functional groups such as, but not limited to, amines, carboxylic acids, hydroxyls, thios, and combinations thereof. The linker can include compounds such as, but not limited to, DTPA, EDTA, DOPA, EGTA, NTA, and combinations thereof. In an embodiment, the linker and the chelator compound are the same, but in other embodiments they can be different. The percentage of linkers attached to the chelator compound, contrast agent, and/or anther linker can be about 0.1 to about 100%.

### Examples 1 and 2

Now having described the embodiments of the of the present disclosure, in general, examples 1 and 2 describe some additional embodiments of the of the present disclosure. While embodiments of the present disclosure are described in connection with examples 1 and 2 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

### Introduction

Nanometer-sized particles such as luminescent quantum dots (QDs) and energy-transfer nanoparticles have unique optical, electronic, and structural properties (e.g., signal brightness, photostability, and multicolor light emission) that are not available from traditional organic dyes and fluorescent proteins. This example describes the use of color-coded nanoparticles and dual-color fluorescence correlation for real-time detection of single native biological targets and viruses in a microfluidic channel. Using green and red nanoparticles to simultaneously recognize two binding sites on a single target, we show that individual molecules of genes, proteins, and intact viruses can be detected and identified in complex mixtures without target amplification or probe/target separation. Real-time correlation analysis of single photon events allows rapid detection of bound targets at 500-1000 data points per second as well as efficient discrimination of excess unbound probes. Quantitative studies indicate that the counting results are remarkably precise and reproducible when the total number of counted molecules is more than 10. When combined with innovative single-molecule approaches, the novel properties of nanoparticle probes open new opportunities in molecular diagnostics, bioterrorism agent detection, and intracellular single-molecule imaging.

This example describes the use of bioconjugated nanoparticles and two-color fluorescence correlation for real-time detection of single native biological targets and intact viruses in a flow channel. Recent research has shown that nanometer-sized particles such as semiconductor quantum dots (QDs) can be covalently linked with biorecognition molecules such as peptides, antibodies, or nucleic acids for use as fluorescent probes. In comparison with organic fluorophores and fluorescent proteins, quantum dots and related nanoparticles exhibit unique optical and electronic properties such as size- and composition-tunable fluorescence emission, large absorption coefficients, and improved brightness and photostability. This example describes a nanoparticle “sandwich” assay in which two nanoparticle probes of different colors simultaneously recognize two binding sites on a single target molecule. This two-site sandwich method relies on a “double-selection” process to improve detection sensitivity and specificity. In this format, target molecules do not need to be chemically derivatized, but the bound targets must be differentiated from excess probes.

In addition, this example illustrates a photon correlation scheme for analyzing the arrival times of single photons on two simultaneous channels. In contrast to photo-burst analysis post-acquisition, this scheme allows real-time detection of single native biological targets and viruses as they flow through a capillary tube. A result is that energy-transfer nanoparticles (embedded with one or more donor/acceptor dye pairs for fluorescence resonance energy transfer or FRET) are excellent probes for single-molecule counting because two or more colors can be excited with a single light source, similar to the case of semiconductor QDs.

### Results and Discussion

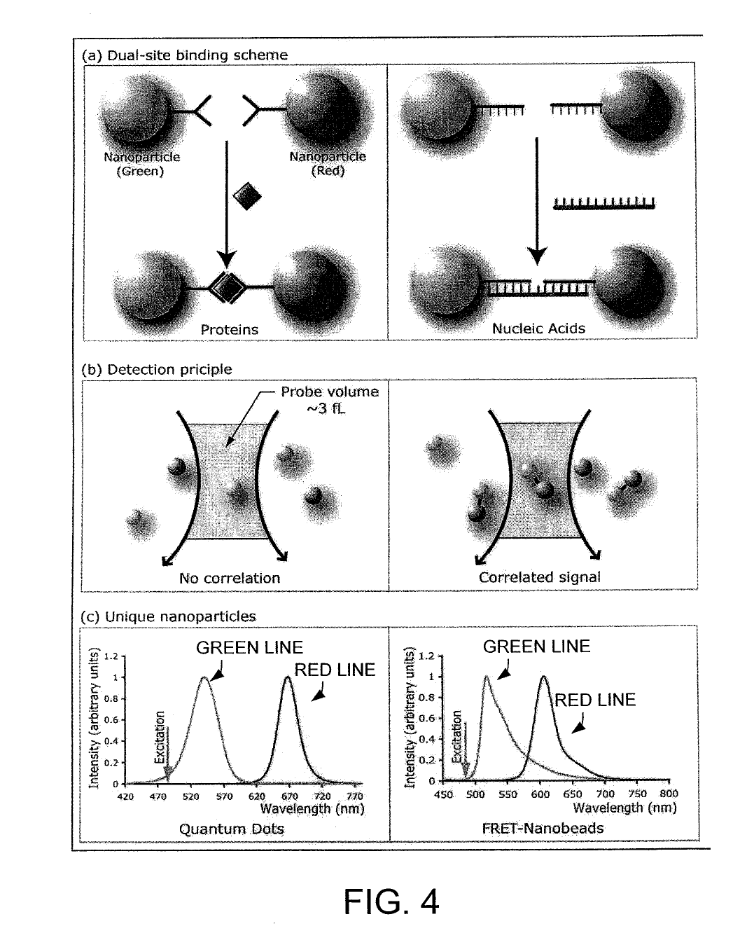

Detection Principles. The basic principles of single-molecule counting using nanoparticle probes are shown in FIG. 4. In this scheme, two bioconjugated nanoparticles are designed to recognize the same target molecule at two different sites (antigenic sites or nucleic acid sequences). This sandwich-type binding brings two color-coded nanoparticles together to form a nanoparticle pair (FIG. 4(a)). This pair moves in solution as a single complex, and when excited by a laser beam, emits green and red fluorescence light simultaneously (i.e., time correlation). In contrast, unbound green and red particles move in a random manner and are unlikely to pass through the laser beam at the same time (FIG. 4(b)). Thus, correlated green and red light emission allows one to discriminate the bound targets from excess unbound probes in a mixture solution.

Both QDs and energy-transfer nanoparticles are excellent probes for single-molecule detection, and their unique optical properties are shown in FIG. 4(c). A major advantage is that a single light source can be used to excite two or more fluorescence colors. A single excitation beam produces only one probe volume, and this overcomes the difficulties in focusing two color laser beams into a small confocal volume (femtoliter or 10⁻¹⁵ L). Others have shown that the co-focusing of two laser beams (par-focality) is an exceedingly difficult task due to both chromatic and spherical aberrations of the microscope objective. Even under carefully matched conditions, the volume overlap for two excitation laser beams was less than 30%. QDs and FRET-nanoparticles also have more symmetric and narrower emission spectra than single-color organic fluorophores, which helps to reduce spectral overlaps between two or more colors.

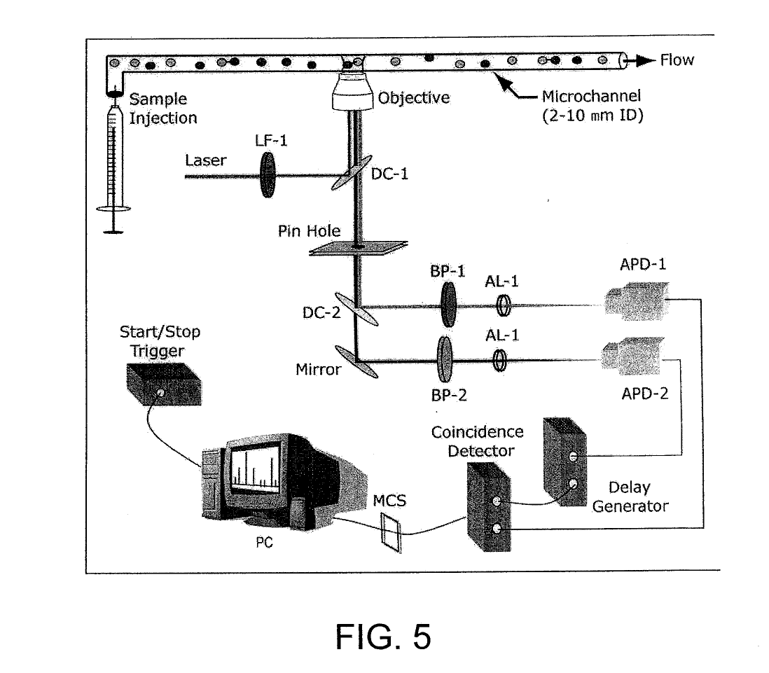

Single-molecule instrumentation is based on an inverted single-point confocal microscope, equipped with two photon-counting avalanche photodiodes (APD-1 and APD-2), a single-photon counting and correlation analysis module (attached to the microscope side port), and a simple capillary flow channel placed on the microscope stage (FIG. 5). In the photon analysis module, fluorescence light emitted from green and red nanoparticles is separated by a dichroic filter (DC-2) and is detected in real time by APD-1 and APD-2. The single-photon output signal from APD-1 is used to trigger a delay generator, which produces a voltage pulse with a controllable width in the range of 200 ns-110 μs. This modified pulse is fed into a co-incidence event detector, and its preset width is used as a time window (called the correlation time) to determine whether one or more photons are detected by APD-2 during this time period. Using a standard photon-counting device such as a multichannel scalar (MCS), the co-incidence output signals are recorded over a period of time. At an integration time of 1-2 ms, this system permits high-speed detection of single molecules in a flow channel at 500-1000 data points per second.

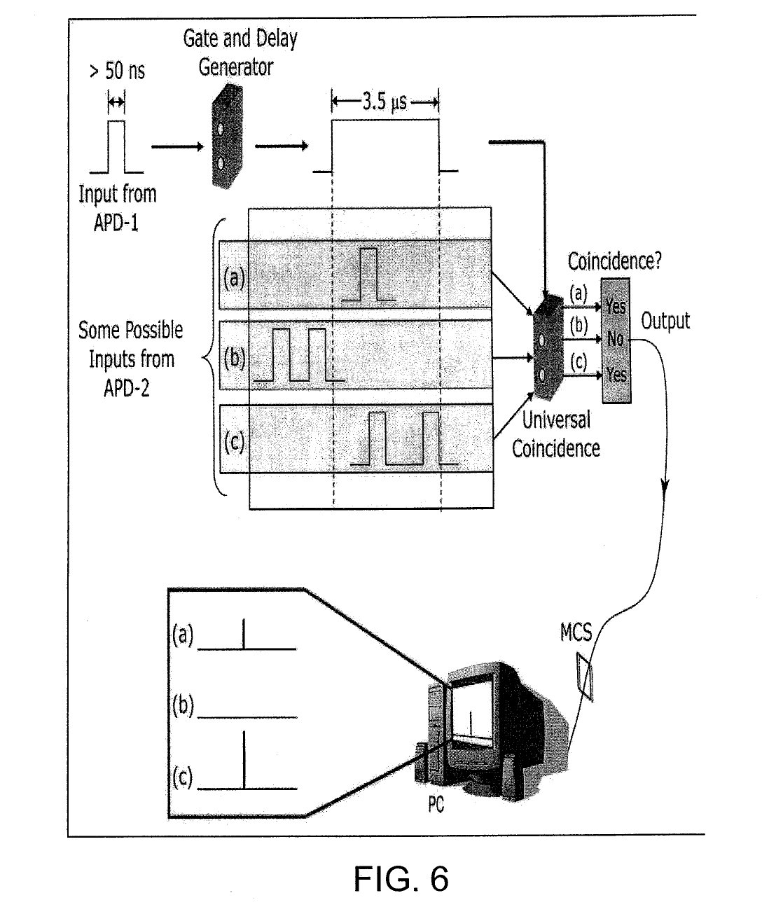

The logic and electronic hardware for real-time correlation analysis of single photons is shown in FIG. 6. This example discusses three representative cases for time-correlated photon detection on two APD detectors. Note that the system is activated by the detection of a single photon on APD-1, which generates a 50-ns wide pulse of 2 volts in amplitude and is converted to a new voltage pulse with a controlled width (typically 3.5 μs, see below). In the first case (a), a single photon is detected by APD-2 during this 3.5 μs correlation time, and the co-incidence detector output is “1”. In the second case (b), no photons are detected by APD-2 during this time window, the co-incidence output is “0”. In the third case (c), two separate photons are detected by APD-2, the co-incidence output is “2”. The co-incidence output signals are recorded over a period of time at a bin or integration time of 1-2 ms for each data point. This allows real-time detection of single molecules based on time correlation of green and red nanoparticle signals.

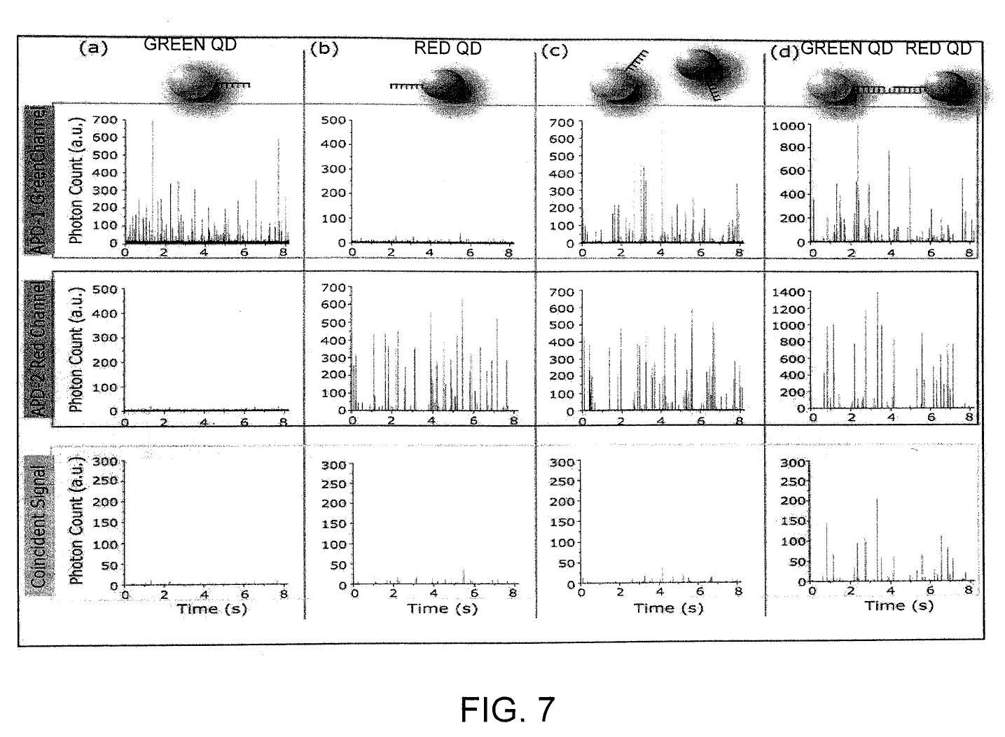

Single DNA Molecules. In addition to real-time recording of the co-incidence signals the system of the present disclosure permits simultaneous data recording from the individual APD channels. FIG. 7 shows real-time photon burst data obtained from APD-1, APD-2, and the co-incidence detector for DNA-nanoparticle bioconjugates in a capillary flow. For green coded nanoparticles, intense single-particle signals are detected only on the green channel (APD-1), while for red-coded nanoparticles, intense signals are only detected on the red channel. For a sample of mixed green and red nanoparticles, both green and red signals are observed, with only weak correlation signals from the co-incidence channel. Significantly, when a target DNA is used to hybridize to both probes, strong correlation signals are detected on the co-incidence channel.

It is worth noting that the co-incidence signals are weak but detectable from the data of individual green and red nanoparticles as well as their mixtures. For single-color particles, background correlation signals arise from residual spectral overlaps between the green and red channels; that is, fluorescence signals in the red channel can “leak” into the green channel and vice versa. For the nanoparticle mixtures, background correlation arises not only from spectral overlap, but also from random events in which a green particle and a red particle happen to pass the probe volume at the same time. Based on Poisson statistics, the probability of finding a red particle at 4 pM concentration in a 3 fL volume is approximately 0.72%, and that of a green particle at 7 pM in a 3 fL volume is 0.1.26%. The probability for finding a red particle and a green particle simultaneously in this volume is 0.009%, which is unlikely to cause a major background problem. Regardless of the dominant source of false correlation signals, the results in FIG. 7 provide a threshold value (˜50 counts/ms) for subtracting “false-positive” events from the true correlation signals in (d). As discussed below, the threshold values for false correlation are dependent on the specific experimental conditions, and can be determined for each nanoparticle system.

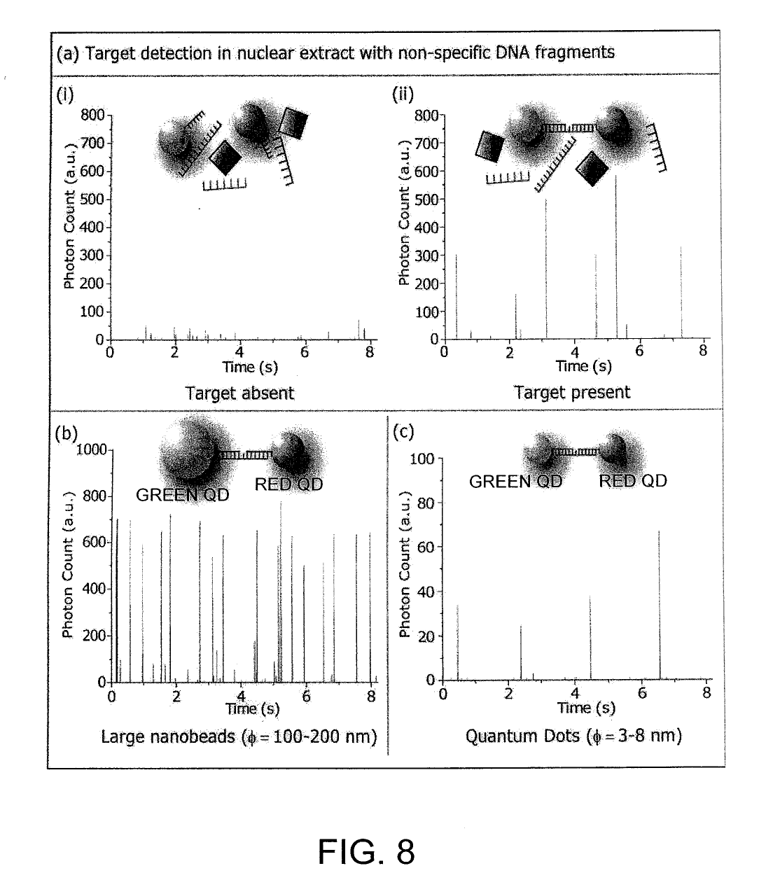

To investigate whether this counting method is able to discriminate against a large excess of unspecific DNA, the green and red nanoparticle probes were mixed with a nuclear extract of Raj i-B cells, and co-incidence signals were recorded with and without a complementary target (FIG. 8(a)). The results reveal that co-incidence signals are highly specific to complementary target DNA, and are not affected by the complex environment of cell lysates (containing nucleic acids, proteins, lipids, and other biomolecules). Furthermore, whether larger energy-transfer particles and smaller QDs could be used for two-color single-molecule detection was investigated. Based on the excellent correlation results in FIGS. 8(b) and (c), it can be conclude that different sized particles in various formats can be used for counting single molecules and for discriminating excess unbound probes in homogeneous solution.

In a systematic effort, several factors have been examined to evaluate and optimize the performance of single-molecule counting. First, the optimal correlation time is dependent on the photon emission rates of the nanoparticle probes and the specific experimental conditions. If this time window is too short, the co-incidence detection clock will be reset before a photon is detected on APD-2, leading to a decrease or loss of correlation signals. If this window is too long, even random photons would be detected as correlation signals, leading to a high background. Under experimental conditions (e.g., the rates of photon emission and 4-5% overall efficiencies of photon detection), statistical calculations suggest that the best signal-to-noise ratios are achieved with a correlation time of 1-5 μs. Experimental measurements of the specific and nonspecific correlation signals also indicate that the best results are obtained with a correlation time of 3-4 μs. Thus, for most single-molecule detection studies the correlation time was set to 3.5 μs.

Second, each nanoparticle should contain at least one ligand for target binding, but too many ligands per particle could lead to aggregation. This kind of aggregation can cause an “under-counting” problem because multiple target molecules in a single aggregate will be counted as one molecule. This problem can be minimized by using “monovalent” probes (that is, each particle is conjugated to approximately one ligand). In this work, nearly monovalent probes (˜1.2 ligands per particle) were prepared and worked at low probe and target concentrations. Third, in the absence of a hydrodynamic focusing mechanism, the nanoparticle probes are expected to follow random trajectories when moving across the laser beam, leading to path-dependent fluctuations in the signal intensity. To correct for this problem, the excitation laser beam should completely fill or cover the capillary tube; that is, the laser beam cross section should be equal or larger than the channel cross section. However, the confocal laser beam used in this example is smaller than the 2-μm channel, and can only detect about ca. 70% of the passing particles, which can be a source of errors leading to target under-counting. Also, it should be noted that the particle flow speed in 2-μm capillaries is approximately 0.55 mm/s (measured experimentally, see methods section). At this rate, it takes 0.5 ms for a particle to move across the laser beam waist (0.262 μm) and 4.3 ms to move through the defocused beam at a region near the channel wall. These transit times indicate that the integration times for each data point should be longer than 0.5 ms but shorter than 4.3 ms, in agreement with the 1-2 ms integration times used in this work.

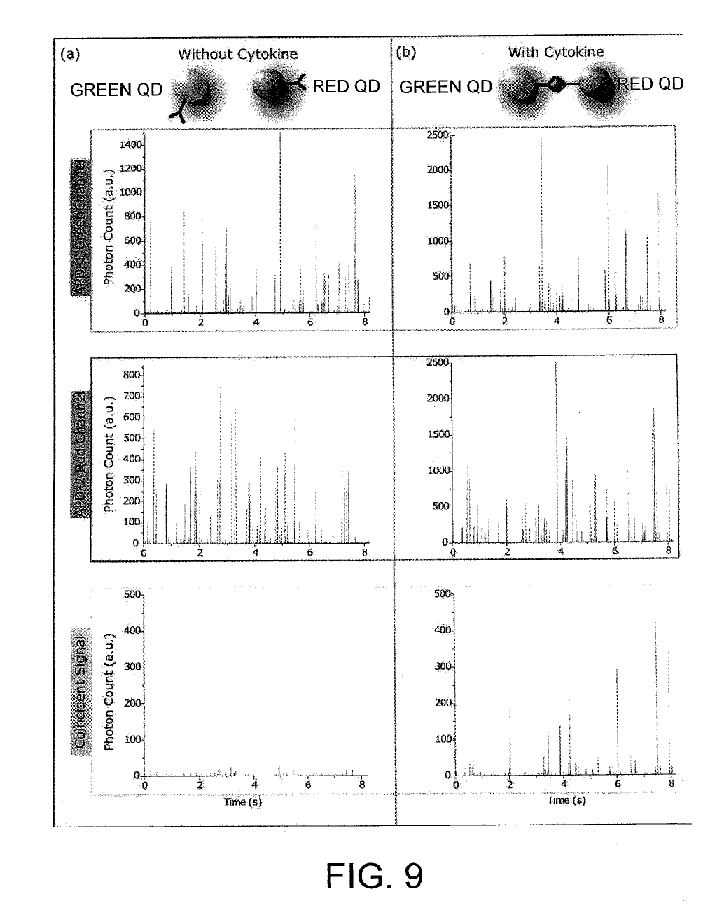

Single Protein Molecules. The above insights obtained from DNA detection also apply to protein detection using two-color nanoparticle probes. FIG. 9 shows real-time photon burst data obtained from two monoclonal antibody-conjugated nanoparticle probes with or without a protein target (50 pg/ml murine TNF-α factor, MW=17 kDa). Similar to two-site DNA hybridization, the target protein simultaneously binds to two nanoparticle probes and generates co-localized fluorescence signals. Also, the control data (without the target protein) yield a threshold value for subtracting the background correlation. A problem in protein detection, however, is that antibody-antigen interactions have finite dissociation equilibrium constants (k), on the order of 10⁻⁹ to 10⁻¹² M. Using second-order binding kinetics as a first approximation, an excess probe concentration equal to k leads to 50% target binding at equilibrium. To achieve 90% target binding, the excess probe concentration is preferred about 9 times of k. This simple analysis suggests that significant probe excess can be used to drive protein binding. On the other hand, excess probes can also cause more background noise, and thus interfere with single-molecule measurement. Based on the level of nonspecific background signals with cytokine antibodies, it was estimated that the maximum probe concentrations might be increased to about 10-20 times of the equilibrium constant k to achieve 90-95% target binding.

An alternative solution is to mix the probes and targets at high concentrations, and then dilute the mixture just before analysis. In this case, the stability of the complex becomes an important factor. Others have found that the dissociation rates for the IgG antibody-antigen complex are about 3×10³ s⁻¹, and that the dissociation equilibrium constants for the antigen-antibody dimmer Ag-Ab₂ are about 2.3×10⁻²¹ M². Under these conditions, quantitative measurement of protein-antibody complexes were reported over 3 orders of magnitude. The achieved sensitivity was limited by the slow rate for a target molecule to encounter the probe laser beam by random diffusion. For example, at 50 fM concentration, the probability for a target molecule to diffuse into in a 2 fL volume is only 0.006%; that is, a target molecule will stay in the laser beam for only one millisecond in a data acquisition period of 15-20 s. To increase the probability of target/laser encounters, the laser beam can be rapidly scanned across the sample (searching for targets), or a microfluidic device can be used to rapidly flow the sample through the laser beam. In the current work, the flow approach was used to increase the number of counted target molecules per unit time. It is worth noting that the total sample volumes to be consumed with micrometer-sized channels do not exceed a few nanoliters during the single-molecule counting experiments. This is particularly useful for manipulation and analysis of small-volume samples such as single cells.

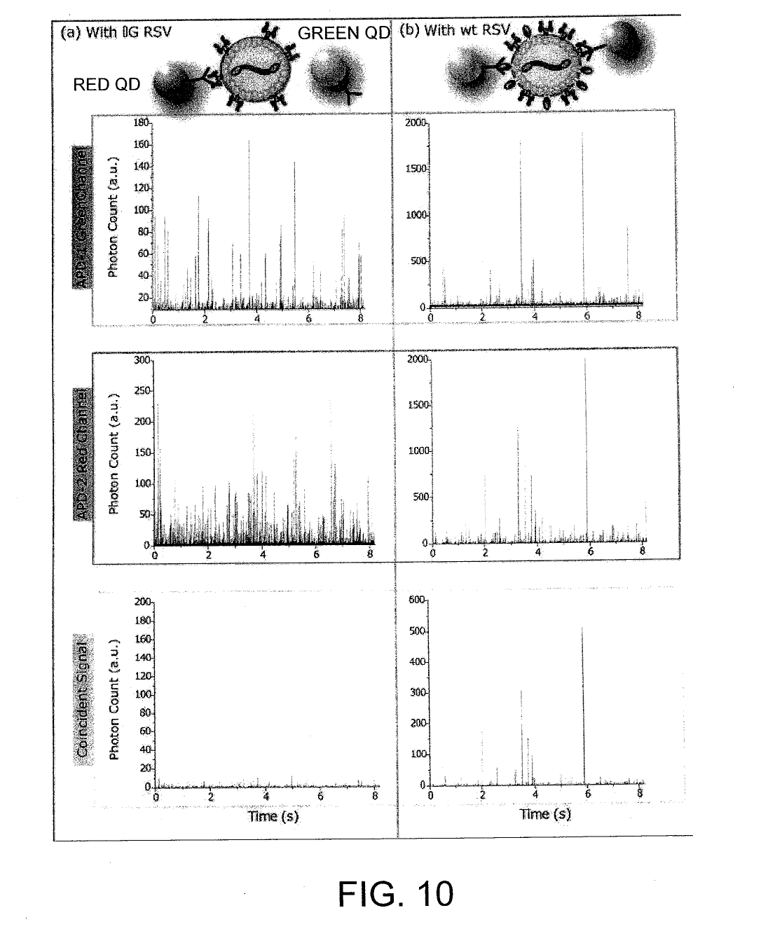

Single Intact Viruses. By using proper and specific antibodies, bioconjugated nanoparticles also allow rapid and sensitive detection of single intact viruses in real time. For example, we have detected single respiratory syncytial virus (RSV) particles in a homogeneous sample. This virus causes serious lower respiratory tract illness in infants and young children, and is a significant pathogen of the elderly and immune compromised. Rapid and sensitive RSV diagnosis is vital to controlling nosocomial infections and for ambulatory and long-term care patients, but current methods are limited by assay sensitivity and slow response. FIG. 10 shows photon-correlation results obtained from the wild-type RVS (with both F and G proteins expressed on the virus surface) and a mutated virus RSV μG (with the G protein deleted). Intense correlation signals are detected from the wild-type viruses because both the green and red probes bind to the F and G viral proteins, respectively. The signals on all three channels are abnormally high, indicating that a single virus has many copies of F and G proteins and is thus able to bind to many green and red particles. In comparison, the mutated virus gives little or no correlation signal because the G protein is not available for binding to green particles. It is also interesting to note that the photon burst intensities on the red channel (F protein) are considerably lower than that of the wild-type virus, suggesting that the level of F protein expression is much lower on the mutated virus than on the wild-type virus.

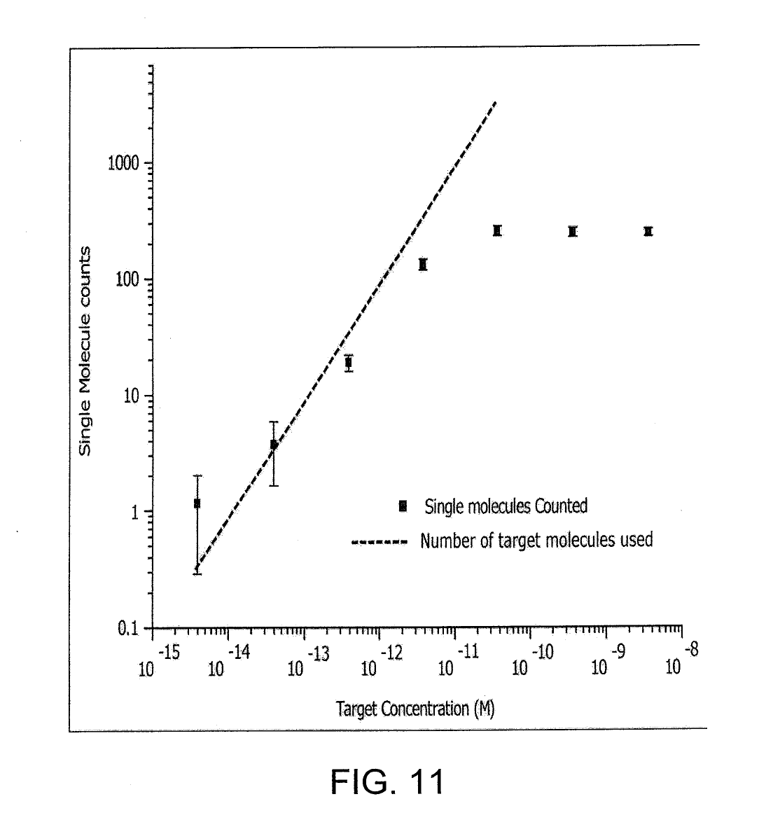

Detection Limit and Quantification. To evaluate the detection limit and the quantitative aspects of this nanoparticle counting technology, target DNA concentration studies were carried out over 6 orders of magnitude (fM to nM) while keeping the probe concentrations constant. At each target concentration, the number of target counts was recorded in a period of 80 seconds, and was repeated 10 times. This gives an average count and a standard deviation for each concentration. FIG. 11 depicts the single-molecule counts as a function of target concentration. The small error bars indicate quantitative and reproducible counting of single molecules, except when the total number of counts falls below 10 (due to statistical or shot noise). Since both the target concentration and the sample flow rate are known, we calculate the actual number of target molecules that flow through the capillary in an 80-second period (dotted line). A comparison with the experimental data indicates that about 40% of the molecules are detected, below the 70% value derived from the expected cross-sectional area overlap between the laser beam and the capillary tube.

An interesting fining is that the number of detected molecules reaches a peak and then slowly decreases with increasing target concentration. This decrease is similar to the “hook effect” in sandwich-type immunoassays, because excess targets cannot generate more sandwich pairs but can cause a breakup of double-binding pairs (Ab₁-Ag-Ab₂) to single-binding complexes (Ab₁-Ag and Ag-Ab₂). The single-binding complexes only show single-color signals, and are not detected by the co-incidence detector. At the low concentration end, the detection sensitivity is determined by statistical noise, that is, the number of molecules that could be counted in a reasonable period of time. With a data acquisition time of 80 seconds and 2-μm flow channels, the present data indicate that single molecules can be detected from target concentrations as lows as 20-30 fM.

A major advantage of single-molecule detection is that target molecules do not need to be amplified for analysis. In current techniques using biochips and polymerase chain reactions (PCR), small samples are not analyzed directly, but are amplified to create a final product for measurement. The amplification process can increase the target materials as much as a million-fold, but it also can introduce errors and uncertainties. Unlike the relative gene or protein expression information obtained from microarrays, single-molecule counting can provide a way to make absolute measurements, which are required for clinical analysis of biomolecular markers in disease detection and diagnosis. For this application, it is important to note that the nanoparticle counting technology of the present disclosure does not require the color-coded nanoparticles to couple or interact with each other; it only requires them to spatially colocalize or correlate within the optical diffraction limit (λ/2 or about 250 nm), and is thus broadly applicable to genes, proteins, and viruses. Furthermore, multiplexed single-molecule counting will be possible with spectrally encoded nanoparticles, allowing a panel of 5-10 biomolecular markers to be analyzed in body fluids (e.g., blood serum). In comparison, other signal amplification methods using nanoparticle biobarcodes and surface plasmon resonance nanosensors do not automatically operate in the single-molecule digital mode.

In summary, a new single-molecule counting method was studied based on the use of nanoparticle probes and two-color fluorescence correlation. The results demonstrate real-time detection of single genes, proteins, and intact viruses in a microfluidic flow channel. The target molecules do not need to be derivatized chemically with a fluorophore, and do not need to be amplified with PCR or other means. Single-molecule counting is achieved in complex mixtures, without washing or separation. In addition, both semiconductor QDs and energy-transfer nanoparticles were found to be bright and stable nanoprobes well suited for simultaneous excitation with a single light source. This molecular counting technology should have broad applications in ultrasensitive medical diagnostics, infectious agent detection, and intracellular single-molecule imaging (in which excess probes cannot be separated or washed away).

### Methods

Instrumentation. Single-molecule instrumentation was based on an invited epi-fluorescence microscope, equipped with two photon-counting avalanche photodiodes (APD-1 and APD-2), and a single-photon counting and correlation analysis module attached to the microscope side port, and a simple capillary flow channel placed on the microscope stage (see FIG. 5). The photon counting and analysis module consisted of (i) one 100-μm pinholes (Melles Griot, Irvine, Calif.), (ii) two high-performance optical filters (Omega Optical Inc., Brattleboro, Vt.), (iii) two aspheric focusing lenses (f=11 mm, NA=0.25, Thorlabs, Newton, MJ), (iv) two photon-counting detector (SPCM-200, EG&G, Vaudreuil, Canada), and (v) two XYZ translation stages (Newport Corp., Irvine, Calif.). A fused-silica capillary with diameters of 2.0 μm (inner) and 150.0 μm (outer) (Polymicro Technologies, Phoenix, Ariz.) was mounted onto an x-y mechanical stage of the microscope. A 1-cm window was cleared on the capillary for optical detection. The sample solution was injected into the capillary by a syringe. A continuous-wave Argon-ion laser (Lexel Laser, Fremont, Calif.) was used for excitation. The 488-nm line was reflected by a dichroic beam splitter (DC-1, 505 DRLP, Omega Optical Inc. Brattleboro, Vt.) and was focused into the capillary window through a high numerical aperture, oil-immersion (n=1.518) 100×/1.25 NA objective lens (Nikon Instrument Group, Melville, N.Y.). Fluorescence light was collected through the same objective, passed through a dichroic beam splitter (DC-1, 505 DRLP, Omega Optical Inc., Brattleboro, Vt.) and a 100 μm pinhole (Melles Griot Co, Irvine, Calif.), and separated by another dichroic beam splitter (DC-2, 560 DCLP, Chroma Technology Corp, Brattleboro, Vt.). After passing through a band-pass filters (BP-1/HQ514 M10 or BP-2/D670 M40, Chroma Technology Corp, Brattleboro, Vt.), the green and red signals were separately focused on two avalanche photodiodes (APD-1 and APD-2) (SPCM-AQR-13, EG&G Canada, Vaudreuil, PQ, Canada) for single-photon detection.

Single-photon events were detected as voltage pulses with a width of ˜50 ns and 2-volts amplitude. The APD-1 output was fed to a delay generator (Model 416a, ORTEC, Oakridge, Tenn.), which extended the pulse width to a controllable value in the range of 200 ns-110 μs. The delay generator output and the ADP-2 output were fed into a universal coincidence unit (model 418a, ORTEC, Oakridge, Tenn.). The co-incidence output was counted by using a multi-channel scalar (EG&G Canada, Vaudreuil, PQ, Canada). At an integration time of 1-2 ms, this system permits high-speed detection of single molecules in a flow channel at 500-1000 data points per second. The principles of real-time correlation analysis of single photon events were further illustrated in FIG. 6.

Nanoparticle Bioconjugation. Oligonucleotides (oligos) or antibodies were covalently conjugated to nanoparticles by using standard cross-linking procedures. For energy-transfer nanoparticles (40 nm) and quantum dots (3-5 nm), the number of oligo or antibody ligands per particle was approximately one (determined experimentally by using fluorescently labeled oligos or antibodies, see below). These nearly “monovalent” nanoparticles produced excellent binding results while avoiding the problem of target/probe aggregation. Thus, a solution of green nanoparticles (40-nm, λₑₓ=505 nm, λₑₘ=515 nm, 53 picomol) (Molecular Probes, Eugene, Oreg.) was mixed with oligo-1 (SEQ ID NO: 1,5′-CTTCAGTTTCTCGGG-A₁₀-NH₂, 52.8 picomol) or oligo-2 (SEQ ID NO: 2,5′-NH₂-A₁₀-CTCCTCCAGCTCCTT-3′, 37.4 picomol) (Sigma Genosys, Woodlands, Tex.), together with an amine activation reagent (50 μg, sulfo-NHS, Pierce, Rockford, Ill.) in a pH=5.75 buffer (0.1 M morpholino ethanesulfonic acid). To this mixture was added a carbodiimide cross-linking agent (50 EDAC, Pierce, Rockford, Ill.). The reaction mixture was thoroughly mixed and sonicated during a two-hour period at room temperature. Bioconjuagted nanoparticles were purified by centrifugation using S-400 microspin columns (Amersham Bioscience, Piscataway, N.J.) at 740 g. Similar protocols were used to conjugate amine-modified oligos to other sized beads as well as semiconductor quantum dots. The bioconjugation efficiencies were determined by using fluorescently labeled amine-oligos (SEQ ID NO: 2, amine- A₁₀-CTCCTCCAGCTCCTT-Oregon Green, Sigma Genosys, Woodlands, Tex.) and plain (nonfluorescent) 40-nm beads. After the cross-linking reaction, free unreacted oligos were separated from the conjugated oligos by centrifugation. Fluorescence intensity measurements of the two fractions indicated that approximately 11% of oligos was conjugated to nanoparticles under our experimental conditions. Similar measurements were used to determine the number of conjugated ligands per nanoparticle.

The same protocols were used for antibody conjugation, such as monoclonal antibodies to the rat anti-mouse/rat tumor necrosis factor-α (TNF-α) (PharMingen/BD Biosciences, San Diego, Calif.). Antibody conjugated nanoparticles were blocked by using PBN buffer (0.5% bovine serum albumin (BSA, Sigma, St. Louis, Mo.) and 0.02% sodium azide in phosphate-buffered saline (PBS, Sigma, St. Louis, Mo.)), and were purified by centrifugation with S-300 microspin columns (Amersham Bioscience, Piscataway, N.J.) at 740 g. For respiratory syncytial virus (RSV) detection, two mouse monoclonal antibodies (Clones 131-2G and 131-2A) (provided by Centers for Disease Control and Prevention, Atlanta, Ga.) were conjugated to green and red nanoparticles, respectively. The coupling and separation procedures were the same as above.

Procedures. DNA hybridization was performed using 4×SSC—sodium dodecyl sulfate (SDS) (1×SSC: 0.15 M sodium chloride and 0.015 M sodium citrate, pH 7.0) for 30 min at 45° C. For cellular lysate studies, the original cell extract (9 mg/ml) was diluted 100 times. For control studies, several noncomplementary targets (total concentration=3 pM) were added into the lysates. For specific targeting binding studies, a complementary target (SEQ ID NO: 3, 2 pM, 5′-CCCGAGAAACTGAAGAAGGAGCTGGAGGAG) was also added to the lysates. These samples were mixed with green probes (SEQ ID NO: 1, 7.2 nM, 5′-CTTCAGTTTCTCGGG-A₁₀-3′-nano) and red probes (4.2 nM, SEQ ID NO: 2, nano-5′-A₁₀-CTCCTCCAGCTCCTT), followed by hybridization at 45° C. for 30 min.

Target protein TNF-α was determined by using sandwich immunoassay: 10 μl of green particles (2.8×10¹³ particles/ml) with the capture antibody was mixed with TNF-α (0.5 μl at 10 μg/ml, R&D System Inc., Minneapolis, Minn.) in 84.5 μl PBN buffer. The mixture was incubated for 45 min at room temperature. To this mixture, a second biotinylated monoclonal antibody (3 μl at 25 μg/ml concentration) was added, followed by an incubation period of 30 min. Then, streptavidin-coated red nanoparticles (ζₑₓ=488, λₑₘ=645 nm, 40-nm diameter, 2 μL of 1.4×10¹⁴ particles/ml) (Molecular Probes, Eugene, Oreg.) were added and allowed to incubate for 30 min. The final binding mixture was diluted 1,000 times before single-molecule analysis.

Both mutated and wild-type RSV viruses were determined by using the following procedure: green probes coupled with purified mouse anti-G monoclonal antibody (Clone 131-2G, 2.8×10¹³ particles/ml, 2 λL) and red nanoparticles coupled with purified mouse anti-F monoclonal antibody (clone 131-2A, 2.8×10¹³ particles/ml, 5 λL) were mixed with the viruses in 250 μl phosphate-buffered saline (PBS)—TBN buffer (0.02% Tween 20, 0.1% bovine serum albumin, 0.02% azide). Binding of nanoparticle probes to virus surface proteins was allowed to proceed at 37° C. for 2 h. The mixture was then diluted 200 times before analysis.

For single-molecule analysis, samples were loaded into a capillary microchannel by microsyringe injection. The flow speed (0.55 mm/s) was determined and calibrated by measuring the time for the liquid to travel a known distance on the miscroscope stage.

Reagents. Nuclear extract and lysates of Raji B-cells (35) were kindly provided by Dr. Uma Nagarajan of Emory University. Except as noted, all chemicals and biochemicals were obtained from commercial sources. Poly-L-lysine hydrobromide (MW=350,000), Trizma base (tris(hydroxymethyl)aminomethane), Trizma hydrochloride (tris(hydroxymethyl)amino-methane hydrochloride), 2-mercaptoethanol, 1-ethyl-3-(3-dimethyl-aminopropyl)-carbodiimide (EDAC), coliphage T4 DNA, λ-DNA, T4 DNA ligase, histone protein and bovine serum albumin were purchased from Sigma Chemical Co. (St. Louis, Mo.). 40 nm carboxylate-modified FluoSpheres were purchased from Molecular Probes (Eugene, Oreg.). Microscope cover slips (0.13 mm thick) were purchased from Fisher Scientific (Pittsburgh, Pa.). Ultrapure water was prepared by a Milli-Q purification system (Millipore, Bedford, Mass.).

### Example 2

### Introduction

In this example, it is shown that antibody-conjugated nanoparticles rapidly and sensitively detect RSV and estimate relative levels of surface protein expression. A major development is use of dual-color quantum dots or fluorescence energy transfer nanobeads that can be simultaneously excited with a single light source.

Respiratory syncytial virus (RSV) is the most important cause of serious lower respiratory tract illness in infants and young children worldwide, causing repeat infections throughout life, and RSV may cause serious complications in the elderly and immune compromised patients. RSV generally initiates mild upper respiratory tract infection in young children with infection rates approaching 50% in the first year of life; however, up to 40% of infected children develop serious lower respiratory tract disease with a substantial number of patients requiring hospitalization. No safe and effective RSV vaccine is available, but immunoprophylaxis is available for certain high risk infants, and strict attention to good infection control practices may prevent nosocomial RSV infection. The availability of sensitive diagnostics can signal the need to implement infection control, guide the timing of immunoprophylaxis, and may be used to guide RSV antiviral treatment.

RSV diagnostics for patient management needs to be sensitive, specific, and rapid requiring direct detection of virus, viral antigens, or RNA usually in nasopharyngeal specimens. RSV isolation in cell culture has been considered the reference method, followed by immunofluorescence assay (IFA) or enzyme immunosorbant assay (EIA); however, results from virus isolation studies are not rapidly available for patient management, and IFA and EIA are not sufficiently sensitive to detect infection in a substantial portion of patients. Serological studies require seroconversion, and thus can not be completed during the acute illness, and PCR assays may be affected by template contamination and false positive results. This example describes nanotechnology based on the principles of microcapillary flow cytometry and single molecule detection, SMD, to detect RSV rapidly and with a high sensitivity.

Two types of fluorescent nanoparticles (40 nm carboxylate modified fluorescent nanoparticles (G nanoparticles: 505/515; R nanoparticles: 488/685, Molecular Probes Inc.)), or two types of Quantum Dots (streptavidin-coated Quantum Dots (QDs: 488/605, QDC Corp.) were used). The microcapillary flow system is integrated with a fixed-point confocal microscope with a high-numerical aperture (NA) 100× objective. At low concentrations the photons emitted from the nanoparticles in the confocal probe volume (˜2 femtoliter) are spectrally separated and analyzed for coincidence of time of arrival at two avalanche photodiodes. To detect RSV, 40 nm fluorescent nanoparticles were coupled to either anti-RSV F protein (clone 131-2A, Chemicon International, Temecula, Calif.) or anti-RSV G protein (clone 130-2G, Chemicon) monoclonal antibodies, allowed to interact with RSV, and the photons produced by laser excitation of the fluorophores passing through the confocal probe volume were detected in real-time.

The system operates on the concept that if two nanoparticles are free to move in a solution, photons generated by them will arrive at the detector at different times unless they are bound to the same target. This concept can be used to detect target molecules at low concentrations with high sensitivity and discriminate between aggregate particles. Traditional fluorescent dyes are less suitable as labels because they require excitation at two different wavelengths making it difficult to ensure that all photon signals are generated from the same probe volume. QDs and FRET nanoparticles used in this system are better suited because they can be excited at the same wavelength while emitting at different colors allowing for dual-color, time-correlated detection of single molecules and single virus particles.

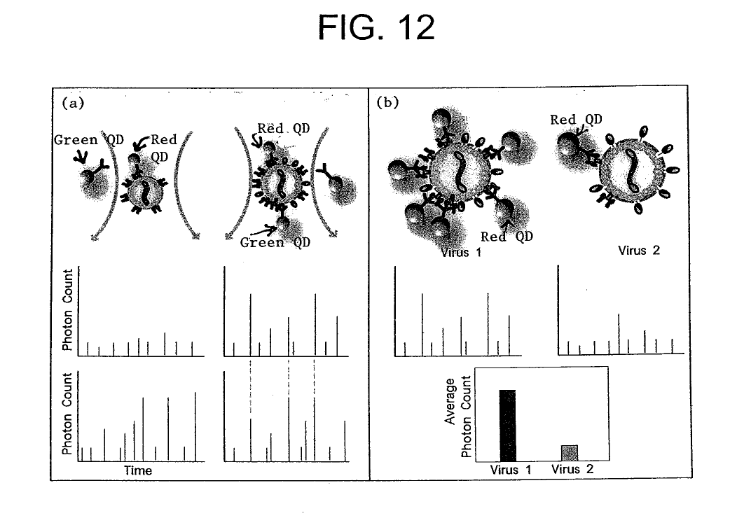

To confirm the specificity of nanoparticle detection, reactivity to uninfected Vero cell lysate, sucrose density purified viruses RSV/A2 and RSVAG (RSV mutant with G protein gene deletion), and parainfluenza virus-type 3 (PIV3) propagated in Vero cells was examined. 10⁵ PFU/ml purified virus was incubated with R nanoparticles (anti-F protein) or G nanoparticles (anti-RSV G protein) as described. The microcapillary flow based SMD system was used to detect very-low levels, and possibly single virus particles, in solution based on the concept that at low concentrations of nanoparticles, coincident photons will be observed only if R and G nanoparticles bind to the same virus particle (FIG. 12(a)). A similar scheme was used to estimate relative amounts of virus surface protein expression (FIG. 12(b)); that is, if one type of virus particle has a higher level of protein expression on its surface, it is likely to bind more of that specific nanoparticle. Hence, the number of photons emitted in the same amount of time will be greater resulting in higher intensity in the integrated photon count spectrum. This concept was used to compare the level of F protein expression on different RSV strains.

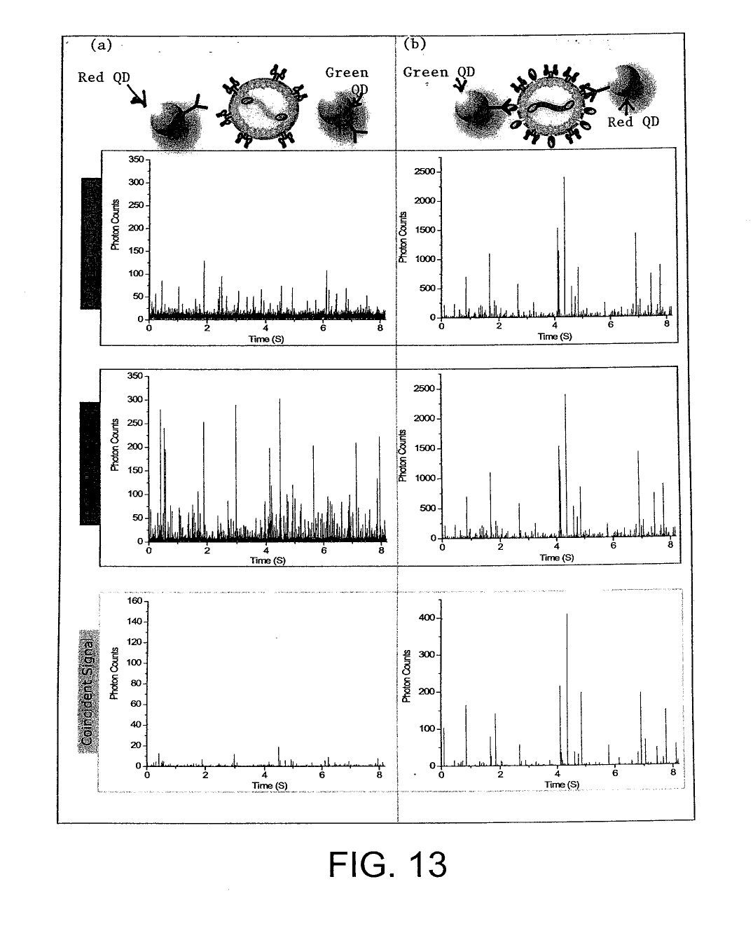

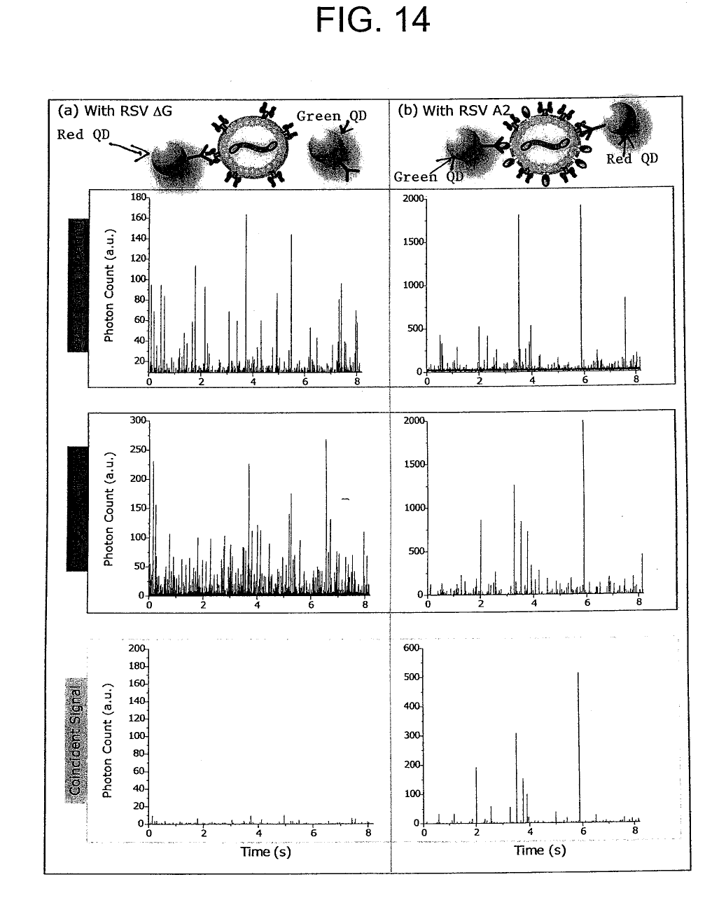

FIG. 13 shows results obtained from photon coincidence experiments for virus particle detection. PIV3 was used as the negative control for nanoparticles conjugated to RSV F or G protein monoclonal antibodies. For PIV3, the photon count signal in both the red and the green channels is low, and there is no coincidence between the signals in the red and the green channels (FIG. 13(a)). In contrast, for RSV/A2 viruses (FIG. 13(b)), there are substantial coincident signals in the red and green channels confirming detection of virus particles. As an additional control, red and green channel signals were examined for detection of RSVΔG (FIG. 14), and did not detect substantial green channel signals or red and green coincidence signals indicating the high specificity of nanoparticle detection. The results in FIG. 13 also indicate that the intensity of photon count signals may be used to estimate the relative level of virus surface protein expression. The increase in photon counts in red and the green channels reflects the number of nanoparticles reactive with virus surface proteins and is independent of coincidence signals. Thus, the photon count in the red or green channel is lower for PIV3 and RSVAG compared to RSV/A2 (FIGS. 13 and 14).

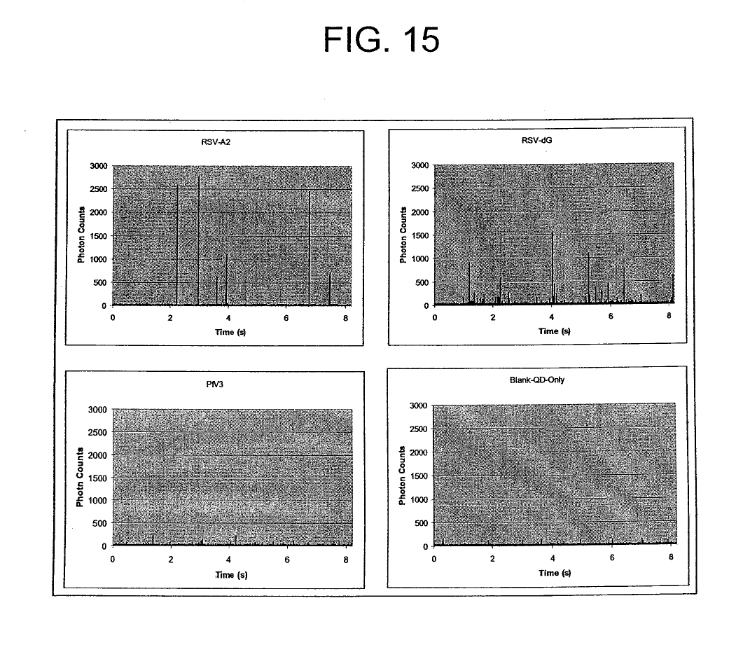

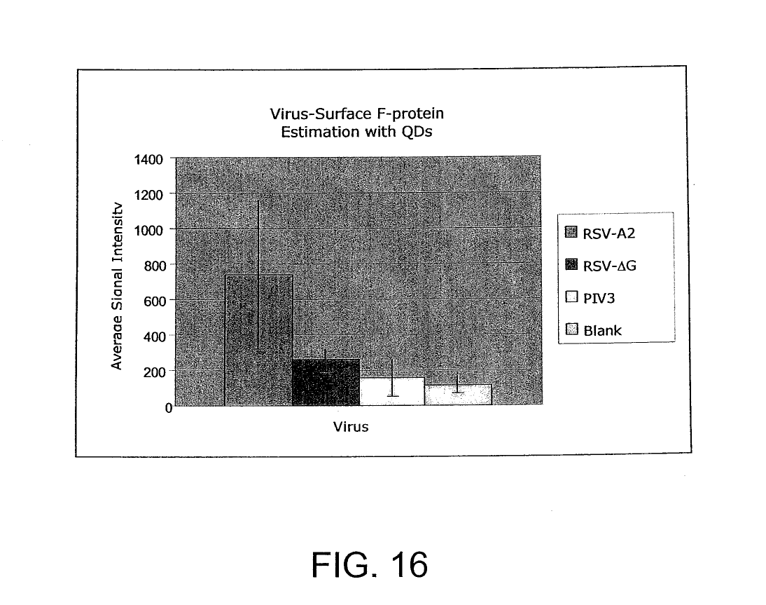

Interestingly, lower levels of R nanoparticle aggregation were detected for the RSVΔG virus compared to the RSV/A2 (FIG. 14). This finding is noteworthy as sequential 3′ to 5′ transcription of the RSV genome is believed to result in higher levels of mRNA transcripts by genes most proximal to the 3′ promoter end, thus higher levels of surface F protein expression might be anticipated by RSVΔG compared to RSV/A2. To address this result, F protein expression on RSVΔG, RSV/A2, and PIV3 using anti-RSV F protein monoclonal antibodies conjugated to QDs was examined at the same concentration used for R nanoparticles detection. A representative experiment of >3 separate experiments is shown in FIG. 15, and the average intensity of photon counts obtained from 35 runs (8 seconds each) is shown in FIG. 16. The intensity average was calculated after removing peaks below 100 counts so that signals from single QDs were eliminated. The average was calculated by dividing total peak intensity by the total number of peaks. As shown in FIG. 15, and similar to the results obtained for R nanoparticles (FIGS. 13 and 14), the hierarchy of peak intensity for QDs that react with RSV F protein is: RSV/A2>RSVΔG>PIV3 blank. It is possible that the lower level of F protein expression by RSVΔG compared to RSV strain A2 may be associated with altered gene expression levels linked to changes in the gene end termination signal that precedes the F protein gene, as changes in this region may affect downstream gene expression levels.

In summary, the present example demonstrates that nanoparticles can be used to rapidly and sensitively detect virus particles, and can be used to estimate the relative amount of surface F protein expression on RSV particles. These features may be useful in multiplexed applications for detecting and differentiating various viruses and other infectious agents in body fluid samples.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

Therefore the following is claimed:

1. A method of detecting a biological target, comprising:
providing a first quantum dot and a second quantum dot, wherein the first quantum dot and the second quantum dot emit energy at different wavelengths, wherein the emission from the first quantum dot is at a first wavelength and the emission from the second quantum dot is at a second wavelength, wherein the first wavelength and the second wavelength are detectably distinguishable, wherein the first quantum dot includes a first biomolecule having an affinity for a first binding site of a biological target, wherein the second quantum dot includes a second biomolecule having an affinity for a second binding site of the biological target, and wherein the biological target includes the first binding site and the second binding site;
mixing the first quantum dot and the second quantum dot with a sample, wherein if the sample includes the biological target, the first quantum dot binds to the first binding site of the biological target and the second quantum dot binds to the second binding site of the biological target to form a first quantum dot/biological target/second quantum dot complex;
exposing the first quantum dot/biological target/second quantum dot complex to a single irradiation source emitting radiation at a single wavelength; and
simultaneously detecting the emission energy from each of the first quantum dot and the second quantum dot, wherein simultaneously detecting the emission from the first quantum dot and the second quantum dot is correlated to the detection of the first quantum dot/biological target/second quantum dot complex.

2. The method of claim 1, wherein the first biomolecule is selected from a polypeptide, a protein, an antibody, an antigen, an aptamer, and a polynucleotide, wherein the second biomolecule is selected from a peptide, a protein, an antibody, an antigen, an aptamer, and a polynucleotide, and wherein the first biomolecule and the second biomolecule are not the same biomolecule.

3. The method of claim 1, wherein the biological target is selected from: viruses, bacteria, cells, microorganisms, micelles, proteins, polypeptides, antibodies, antigens, aptamers, and polynucleotides.

4. The method of claim 1, further comprising:
flowing the first quantum dot/biological target/second quantum dot complex through a channel.

5. The method of claim 4, wherein the channel is a microcapillary channel.

6. The method of claim 4, further comprising:
focusing an irradiation source on a portion of the channel.

7. The method of claim 6, further comprising:
detecting a first signal from the first quantum dot at the first wavelength with a first detector and detecting a second signal from the second quantum dot at the second wavelength with a second detector.

8. The method of claim 7, further comprising:
determining if the first signal and the second signal were received simultaneously.

9. The method of claim 7, further comprising a first filter to filter out wavelengths that are not the wavelength of the first signal, and a second filter to filter out wavelengths that are not the wavelength of the second signal.

10. The method of claim 1, wherein each of the first quantum dot and the second quantum dot are selected from: a quantum dot having an emission at about 525 nm, a quantum dot having an emission at about 585 nm, a quantum dot having an emission at about 605 nm, a quantum dot having an emission at about 655 nm, and a quantum dot having an emission at about 685 nm, and wherein the first quantum dot and the second quantum dot do not have the same emission wavelength.

11. The method of claim 1, wherein the biological target is a virus.

12. The method of claim 11, wherein the biological target is an RSV virus.

13. A method of detecting a biological target, comprising:
providing a first bead and a second bead, wherein different wavelengths of emission energy are emitted from the first bead and the second bead, wherein the emission from the first bead is at a first wavelength and the emission from the second bead is at a second wavelength, wherein the first wavelength and the second wavelength are detectably distinguishable, wherein the first bead includes a first biomolecule having an affinity for a first binding site of a biological target, wherein the second bead includes a second biomolecule having an affinity for a second binding site of the biological target, and wherein the biological target includes the first binding site and the second binding site;
mixing the first bead and the second bead with a sample, wherein if the sample includes the biological target, the first bead binds to the first binding site of the target molecule and the second bead binds to the second binding site of the target molecule to form a first bead/biological target/second bead complex;
exposing the first bead/biological target/second bead complex to a single irradiation source emitting radiation at a single wavelength; and
simultaneously detecting the emission energy emitted from each of the first bead and the second bead, and wherein simultaneously detecting the emission from the first bead and the second bead is correlated to the detection of the first bead /biological target/second bead complex.

14. The method of claim 13, wherein the first bead is a first FRET-bead and the second bead is a second FRET-bead, wherein the first FRET-bead includes a first donor molecule and a first acceptor molecule, wherein the second FRET-bead includes a second donor molecule and a second acceptor molecule, and wherein each of the first donor molecule, the second donor molecule, the first acceptor molecule, and the second acceptor molecule emit radiation energy at distinguishably different wavelengths.

15. The method of claim 14, wherein the first biomolecule is selected from a polypeptide, a protein, an antibody, an antigen, an aptamer, and a polynucleotide, wherein the second biomolecule is selected from a peptide, a protein, an antibody, an antigen, an aptamer, and a polynucleotide, and wherein the first biomolecule and the second biomolecule are not the same.

16. The method of claim 14, wherein the biological target is selected from viruses, bacteria, cells, microorganisms, micelles, proteins, polypeptides, antibodies, antigens, aptamers, and polynucleotides.

17. The method of claim 16, wherein the biological target is a virus.

18. The method of claim 17, wherein the biological target is an RSV virus.

19. The method of claim 14, further comprising:
flowing the first bead/biological target/second bead complex through a channel.

20. The method of claim 19, further comprising:
focusing an irradiation source on a portion of the channel.

21. The method of claim 20, further comprising:
detecting a first signal from the first bead with a first detector and detecting a second signal from the second bead with a second detector.

22. The method of claim 21, further comprising:
determining if the first signal and the second signal were received simultaneously.

23. The method of claim 13, wherein the first bead is a first dye-bead and the second bead is a second dye-bead, wherein the first dye-bead includes a first dye, wherein the second dye-bead includes a second dye, and wherein the first dye and the second dye emit radiation energy at distinguishably different wavelengths.

24. The method of claim 23, wherein the first biomolecule is selected from a polypeptide, a protein, an antibody, an antigen, an aptamer, and a polynucleotide, wherein the second biomolecule is selected from a peptide, a protein, an antibody, an antigen, an aptamer, and a polynucleotide, and wherein the first biomolecule and the second biomolecule are not the same.

25. The method of claim 23, wherein the biological target is selected from viruses, bacteria, cells, microorganisms, micelles, proteins, polypeptides, antibodies, antigens, aptamers, and polynucleotides.

26. The method of claim 23, wherein the biological target is a virus.

27. The method of claim 26, wherein the biological target is an RSV virus.

28. The method of claim 23, further comprising:
flowing the first bead/biological target/second bead complex through a channel.

29. The method of claim 28, further comprising:
focusing an irradiation source on a portion of the channel.

30. The method of claim 29, further comprising:
detecting a first signal from the first bead with a first detector and detecting a second signal from the second bead with a second detector.

31. The method of claim 30, further comprising:
determining if the first signal and the second signal were received simultaneously.

32. A single-molecule detection system comprising:
a channel for flowing a fluid that includes a fluorescent/biological target complex, wherein the fluorescent/biological target complex emits at least two radiation energy signals upon irradiation by a single wavelength of radiation energy, wherein each radiation energy signal is at a distinguishably different wavelength;
an irradiation and optic system including an irradiation source configured to direct a single wavelength of an irradiation beam onto a portion of the channel, wherein the irradiation and optic system is configured to direct the at least two emitted radiation energy signals from the fluorescent/biological target complex to the detector and co-incidence system;
a detector and co-incidence system including at least two detectors, wherein each detector includes a filter that allows only a wavelength for one of the radiation energy signals emitted from the fluorescent/biological target complex to pass through each of the filters, wherein the detector and co-incidence system includes a co-incidence detector interfaced to one of the detectors and a delay generator interfaced to the another detector, wherein the delay generator and the co-incidence detector are directly interfaced, wherein the co-incidence detector is configured to determine if co-incident signals are emitted from the fluorescent/biological target complex; and
an analysis system in communication with the co-incidence detector, wherein the analysis system receives data from the co-incidence detector if the detectors simultaneously receive radiation energy signals emitted from the fluorescent/biological target complex.


SEQUENCE LISTING (3 sequences)

SEQ ID NO: 1 - DNA, 15 bp, Artificial Sequence
  Chemically synthesized DNA sequence for green nanoparticle probe
cttcagtttctcggg

SEQ ID NO: 2 - DNA, 15 bp, Artificial Sequence
  Chemically synthesized DNA sequence for red nanoparticle probe
ctcctccagctcctt

SEQ ID NO: 3 - DNA, 30 bp, Artificial Sequence
  Chemically synthesized complementary DNA target sequence
cccgagaaactgaagaaggagctggaggag